(19) United States
(12) Patent Application Publication
(10) Pub. No.: US 2023/0417786 A1
(43) Pub. Date: Dec. 28, 2023

(54) FULLY AUTOMATED SAMPLE PROCESSING PLATFORM FROM EXTRACTION TO SEQUENCING

(71) Applicant: Clear Labs, Inc., San Carlos, CA (US)

(72) Inventors: Tiffany Cheung, San Carlos, CA (US); Michael Balamotis, San Carlos, CA (US); Werther Dong, San Carlos, CA (US); Alfred Haddad, San Carlos, CA (US); Prasanna Thwar, San Carlos, CA (US)

(21) Appl. No.: 18/340,158

(22) Filed: Jun. 23, 2023

Related U.S. Application Data
(60) Provisional application No. 63/355,478, filed on Jun. 24, 2022.

(51) Int. Cl. G01N 35/10; G01N 35/02

(52) U.S. Cl. CPC G01N 35/10; G01N 35/026; G01N 2035/00346

(57) ABSTRACT

The present disclosure provides for processes and systems that rely on software, bioinformatics, and robotics principles for actuating the automation of a workflow from nucleic acid extraction from a sample to sequencing without human intervention in between the aforementioned steps.

### CROSS-REFERENCE

The present application claims priority to U.S. Provisional Application Ser. No. 63/355,478, filed Jun. 24, 2022; the contents of which are hereby incorporated by reference in its entirety.

### FIELD OF THE INVENTION

The present disclosure relates to systems, software, and bioinformatic pipelines for integrating various sequencing platforms, particularly platforms that use sequencing-by-synthesis (SBS) chemistries, or pore sequencing chemistries, to automated platforms for sample and library preparation. Automation of sample and library preparation, including sample extraction and loading of sequencer cartridges into sequencer machines obviates the need for human intervention previously necessary to transfer libraries prepared for sequencing onto sequencing devices, particularly sequencing-by-synthesis ones.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions. Systems configured to perform sequencing protocols are generally constrained by steps that required substantial manipulation of a reagent or cartridge (e.g., loading of a sequencing module, comprising a sequencing cartridge and sequencing flow cell, into a sequencer). Such systems capabilities may not be cost-effective. Thus, there is a general need for improved systems, methods, and apparatuses that are capable of performing or being used during assay protocols, such as the sample and library preparations described above, in a cost-effective, simpler, or otherwise improved manner.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

In many aspects, the disclosure provides systems, apparatus, liquid handlers, robotic actuators, and workstations actuated by a cloud-based or local software system e.g., (a plurality of software applications networked together via a plurality of application programming interfaces; APIs for automating a workflow from nucleic acid sample preparation to nucleic acid sequencing.

In some aspects, the disclosure provides a system for automating a nucleic acid sequencing process, comprising: a robotic arm having a left effector and a right effector, said robotic arm connected to a rotary axis, said rotary axis connected to an apparatus for actuating one or more programmed movements of the robotic arm, wherein the apparatus is configured for receiving an input from the system for: moving said robotic arm towards a sequencer cartridge on a location; extending the left effector and the right effector of the robotic arm around the sequencer cartridge on the location and subsequently contracting said effectors around the sequencer cartridge, thereby holding the sequencer cartridge with the robotic arm; moving the sequencer cartridge that is held by the robotic arm into a sequencer machine thereby loading the sequencer cartridge; and a sequencer machine for sequencing a sample in the sequencer cartridge. In many instances, the rotary axis is configured for rotating 360° around its own axis. The rotary axis can rotate the robotic arm above the location housing the sequencer cartridge. The left effector and the right effector can be symmetrical or asymmetrical depending on the configuration of the cartridge to be lifted. In preferred cases, the left effector and the right effector are configured for lifting a variety of different sequencing cartridges, which can weigh at least 1 pound, at least 2 pounds, at least 5 pounds, or at least 10 pounds when the flow cell is mounted therein. It is advantageous to have a left effector and a right effector that can lift such weights. In many instances, the sequencer machine is configured for performing sequencing-by-synthesis, see, e.g., Example 2 demonstrating the performance of a fully automated method for robotically preparing samples and robotically loading such samples into a sequencer. In many configurations, the system of the disclosure is configured for providing one or more inputs directly to a sequencer, FIGS. 1 and 2, providing an illustration of how this was executed. In some configurations, the sequencer machine automatically starts a sequencing run upon loading of the sequencer cartridge. In many instances, two or more sequencing machines with different capabilities can robotically receive a sequencing cartridge from an apparatus of the disclosure.

In many aspects, the disclosure provides systems, apparatus, liquid handlers, robotic actuators, and workstations actuated by a software system e.g., (a plurality of software applications networked together via a plurality of APIs) for automating a workflow from nucleic acid sample preparation to nucleic acid sequencing. In many configurations, a software application functionally connected to the system actuates a liquid handling system functionally operating on a multi-module workstation having nucleic acid samples and reagents therein for preparing such nucleic acid samples for sequencing. In particular configurations, the liquid handling system is configured for piercing a cover over the loading port of the sequencing cartridge thus providing a process for loading a nucleic acid sample that has been prepared for sequencing into the flow cell. In many aspects, the liquid handling system is configured to receive an input from a specific software application that informs when the sample has been prepared for sequencing. In some configurations, a specific software application can provide calculations from the cloud-based software systems which calculate the pooling volume required for each sample- to achieve a selected sequencing coverage for at least one selected microorganism genome- and load the pierced sequencing module, comprising a sequencing cartridge and sequencing flow cell, with the library dynamically prepared based on the instructions provided through a separate software application. In many configurations, the nucleic acid sample was prepared for sequencing in the multi-module workstation, e.g., FIG. 3. In many configurations, the nucleic acid sample was robotically prepared for sequencing in the multi-module workstation without human intervention. The disclosure contemplates workstations that comprise a plurality of multi-well plates, chambers, or another suitable system for receiving samples. In some instances the workstation comprises a plurality of racks, e.g., racks holding chambers. In many instances, the plurality of racks include multi-well plates or chambers for holding reagents for extracting a nucleic acid from a cell, e.g., reagents for cell lysis. In many instances, the plurality of racks include multi-well plates or chambers for holding reagents for fragmentation of a nucleic acid, multi-well plates or chambers for holding reagents for end repair of a nucleic acid, multi-well plates or chambers for holding reagents for reverse transcription of one or more nucleic acids, multi-well plates or chambers for holding reagents for amplification of one or more nucleic acids, multi-well plates or chambers for holding reagents for adding a sequencing adaptor to one or more nucleic acids, multi-well plates or chambers for holding reagents for nucleic acid purification. In many configurations, the workstation comprises a magnet plate, a vacuum manifold, and a thermal cycler. In many configurations, the workstation is an open workstation, e.g., the sequencer is outside.

In some aspects, the disclosure provides a cloud-based or local software system comprising instructions which, when the program is executed by a computer, cause the system to actuate the robotic insertion of a cartridge into a sequencer machine. In some aspects, the disclosure provides a process for robotically inserting a cartridge into a sequencer machine comprising using such systems. In some aspects, a clickbot application or another suitable “stand-alone” software application is used to open the sequencer door by integration into the workflow of a system of the disclosure. In other aspects, the software application used to actuate the opening of the sequencer door is fully integrated with a software system code of the disclosure. Systems of the disclosure may comprise an integration of one or more software applications with a software system to actuate movement of robotic parts within the system based on custom inputs provided by a user. A specific software application of the disclosure is, in some aspects, custom designed to calculate certain sample parameters, e.g., volume, concentration, based on a genome coverage size and a genome size selected by a user and adjust the movement of the equipment can be controlled by the a separate software application based on those parameters. In many instances, the software system initiates a custom software application that, e.g., determines an amount of a sample input (e.g., library pooling volume based on WIZARD calculations to meet a coverage chosen per sample) based on one or more characteristics of a selected, e.g. bacterial, fungi, viral, or eukaryotic genomes size and coverage. Such custom input is integrated in a software application to actuate a system of the disclosure and provide a fully automated process for the preparation and sequencing of nucleic acid samples.

In some aspects, the disclosure provides software application for functionally connecting a plurality of commands for robotically automating a nucleic acid sequencing workflow comprising: a non-transitory computer readable medium storing instructions that, when executed by at least one programmable processor, causes at least one programmable processor to perform operations comprising: receiving, by a computing device, a request for analyzing a selected at least one microorganism genome and a selected sequencing coverage for the at least one microorganism genome; inputting a size of at least one selected genome of the microorganism and the selected sequencing coverage into a flow cell capacity bar whereby the flow cell capacity bar is configured to process a percent usage of a flow cell for at least one selected genome and the selected sequencing coverage; outputting a percent capacity of the flow cell capacity bar; and thereby providing a software application for determining the capacity of each sequencing run. The disclosure envisions such software application can be programmed to actuate loading of a variety of flow cells, including sequencing-by-synthesis flow cells and pore sequencing flow cells. In some aspects, a specific software application calculates a genome size based on a reference genome size of a genus or species of at least one genome of the microorganism. In some instances, a specific software application calculates a sample pooling amount for each sample based on the selected sequencing coverage. In preferred instances, the short nucleic acid sequencing reads and long nucleic acid sequencing reads are processed using two different sequencing modalities, independently and within the constraints of each platform in terms of total sequencing depth, quality thresholds, and read length. Short and long read data can then be combined for hybrid de novo sequence assembly. In some aspects, such hybrid sequencing approach overcomes challenges in genome assembly and in placing highly repetitive sequences in a genome. Because depth of coverage is affected by the accuracy of genome alignment algorithms and by the uniqueness or the ‘mappability’ of sequencing reads within a target genome the hybrid approach used, in such instances, was able to predict with greater accuracy the identification of a target genome. In some instances, the hybrid approach was at least 1.0%, at least 20%, or at least 30% more accurate in the identification of a target genome when a same coverage depth was selected.

In other instances, the sample pooling amount is a volume amount from a sample culture. In some instances, the non-transitory computer readable medium further comprises computer instructions for actuating a liquid handling system for loading the sample pooling amount into a rack on a workstation (e.g., a rack can hold a chamber, multi-well plates, or another suitable reagent container) of an apparatus for automating a nucleic acid sequencing process. In some instances, the non-transitory computer readable medium further comprises computer instructions for automatically actuating a robotic system or a liquid handling system for automating a nucleic acid sequencing process from sample preparation to loading into a sequencer upon determining the capacity of each sequencing run. The computer instructions may actuate the liquid handling system to perform one or more of, not necessarily in the order shown below: a) moving a nucleic acid sample into a well or a tube with reagents for extracting a nucleic acid from a cell, e.g., via cell lysis; b) moving a nucleic acid sample into a well or a tube with reagents for purification of the nucleic acid from the cell debris e.g., via magnetic clean up; c) moving a nucleic acid sample into a chamber with reagents for fragmentation of a nucleic acid; d) moving a nucleic acid sample into a chamber with reagents for end repair of a nucleic acid; e) moving a nucleic acid sample into a chamber with reagents for reverse transcription of a nucleic acid; f) moving a nucleic acid sample into a chamber with reagents for amplification of one or more nucleic acids; g) moving a nucleic acid sample into a chamber with reagents for amplification of one or more nucleic acids; amplification of one or more nucleic acids; h) moving a nucleic acid sample into a chamber with reagents for adding a sequencing adaptor to one or more nucleic acids; i) moving a nucleic acid sample into a chamber with reagents for denaturation of one or more nucleic acids; j) moving a nucleic acid sample into a chamber with reagents for hybridization or capture of one or more nucleic acids using probes; k) combining two or more nucleic acid samples from different wells or tubes into a single well or tube; and/or l) for pooling a volume of a nucleic acid sequencing library calculated to meet the selected sequencing coverage. In many instances, the integrated software system (see FIG. 1) provides input for the desired pooling volumes required for a fully automated sequencing run and thus informs the actuation of the robotics by the appropriate software application. The computer instructions actuate the robotic arm for moving a chamber having a nucleic acid sample therein to a magnet plate, to a vacuum manifold in the workstation, to a thermal cycler, or to another chamber in the workstation. A specific software application, e.g., (WIZARD) provides differentiated library pooling calculations based on selected read coverage by sample (e.g., virus, fungi, bacteria, mammalian, or another eukaryotic genome). The integration of various software applications supports the custom sample preparation in the work-deck of a system of the disclosure as well as the automated loading of the library onto the flow cell, the automated cartridge insertion into the sequencer, the automated run parameter and run start (e.g., can use a software application or clickbot application to actuate the opening of the sequencer door for receiving the sequencing module, comprising a sequencing cartridge and sequencing flow cell, or can utilize a fully customized integrated code that directs the opening of the sequencer door), and, in some instances, automatic upload of the sequenced data into the cloud. In some cases, a sequencing data and/or report can be sent directly to a user in FASTQ, FASTA, or another suitable format after the completion of the fully automated sequencing run.

In many aspects, a system of the disclosure is a fully automated system comprising a plurality of software subsystems, software applications, software components, and software modules networked together. The fully automated software system can comprise a plurality of External APIs and a plurality of internal APIs, and it can comprise a plurality of AI software applications and systems. Notably, the fully automated software system commands a plurality of bioinformatics pipelines, including a plurality of analytical and reporting softwares or modules, e.g., a plurality of software applications networked together. In some aspects, the fully automated software application network is integrated with a bioinformatics pipeline and a software application for providing input to the liquid handling system, based on parameters determined by software application to meet desired output parameters from the bioinformatics pipeline functionally connected to the multi-module workstation, including, e.g., the determination of the maximum load size (capacity cell calculation) for each genome to be analyzed. This provides a previously undescribed technical advantage that supports the determination of an input that is a sample pooling volume for a sequencing library. The solution provided in the instant disclosure supports a fully automated system and overcomes many of the existing technical challenges that prevented a full automation of such systems to be previously achieved.

In certain aspects, the disclosure provides a software application (and processes for using the same) comprising instructions which, when the program is executed by a computer, cause the computer to instruct one of the systems described herein to robotically insert a sequencing module, comprising a sequencing cartridge and sequencing flow cell, into a sequencer.

The selected at least one microorganism genome can be a genome of the Escherichia genus, the Listeria genus, the Salmonella genus, or the Campylobacter genus. However, the platform is not limited to these genera of bacteria. It can process any gram-positive and gram-negative bacteria, viruses, a fungi, a parasite(s), a mammalian system(s), or any organism(s) with a sequenced referenced genome.

In some aspects, the disclosure provides a system for controlling an integrated software system by functionally integrating a plurality of commands from a plurality of software applications for robotically automating a nucleic acid sequencing workflow comprising: receiving, by the system, a request for analyzing a selected at least one microorganism genome and a selected sequencing coverage for the at least one microorganism genome; inputting, by the system, a size of the selected at least one genome of the microorganism and the selected sequencing coverage into a flow cell capacity bar whereby the flow cell capacity bar is configured to process a percent usage of a flow cell for the selected at least one genome and the selected sequencing coverage; outputting, by the system, a percent capacity of the flow cell capacity bar, thereby providing a feature for determining a capacity of each sequencing run; and a web application thereby providing a system for user defined sample analysis output metrics. In certain aspects, a single or a series of software application(s) are responsible for tracking sample related information from the inputs to the bioinformatics pipeline output. The single or the series of software application(s) making up the software system can allow for the communication of the automation workflow and for controlling the bioinformatics pipelines.

The system of the disclosure can analyze the genomes of various microorganisms. In some cases, at least one microorganism genome is a virus, e.g., SARS-CoV-2, an influenza A, an influenza B, or Human Respiratory Syncytial Virus (RSV) genome.

These aspects and other features and advantages of the invention are described below in more detail.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1 illustrates an exemplary integration of a software system for determining sample processing parameters with an automated robotics platform for automating a process from sample preparation to sequencing and downstream bioinformatics analysis, based on customer sample input.

FIG. 2 illustrates an exemplary user interface software application for automating a process.

FIG. 3A illustrates an example of a robotic work deck controlled by a software system and bioinformatic pipelines described herein. The work deck is part of a system configured to be connected with a system comprising a liquid handling system for preparing a nucleic acid sample and a robotics arm component for grabbing and loading a sequencing module, comprising a sequencing cartridge and sequencing flow cell, into a sequencer.

FIG. 3B illustrates a rear view of a robotic arm comprising a right effector (316) and a left effector (317) connected to a rotary arm (318). FIG. 3B further illustrates a liquid handling system having eight individual pipet channels (319).

FIG. 3C illustrates a front view of a robotic arm having a right effector (316) and a left effector (317) connected to a rotary arm.

FIG. 3D illustrates a side view of a robotic arm having a right effector and a left effector connected to a rotary arm. FIG. 3D illustrates two rotary cylinders 318A and 318B (collectively with their components they form 318 rotary arm), movement of both of which is controlled by software applications described herein, an schematic of which is shown on FIG. 1.

FIG. 3E illustrates one end of a robotic work deck, operationally connected to a robotic arm and a liquid handler system controlled by software applications described herein. FIG. 3E depicts a conveyor belt functionally mounted to a side support of the robotic work deck for moving the robotic arm (318; collectively comprising cylinders 318B and 318A) and the liquid handler mounting on a movable support (322) backwards and forwards. Eight individual pipet channels (319) in the liquid handler are illustrated.

FIG. 3F illustrates another end of a robotic work deck controlled by the software system described herein. FIG. 3F depicts a robotic arm preparing to load a sequencing module, comprising a sequencing cartridge and sequencing flow cell, (depicted as being grabbed by the robotic arm) into a nucleic acid sequencer (315) that is physically located outside the work-deck (off-deck) (324). The movable support (322) mounted on a side support—which slides backwards and forwards—is depicted in relation to the filter tips 303 and 304 displayed on reagent racks for movable support (322). FIG. 3F illustrates the robotic arm extending beyond the work deck to load a sequencing module (325) into a sequencer 315. Two sequencers can be seen side by side in this illustration.

FIG. 4A illustrates a front view of a sealed loading port of a sequencing cartridge.

FIG. 4B illustrates a front view of one pipet channel from liquid handler 319 loading a sample volume calculated as described in the workflow of FIG. 2 into the sequencing cartridge.

FIGS. 4C and 4D illustrate a front view and a side view of a sequencing cartridge that has received a nucleic acid sample prepared for sequencing, respectively.

FIG. 4E illustrates a side view of one pipet channel, with a filter tip attached, from liquid handler 319 loading a sample volume calculated as described in the workflow of FIG. 2 into the loading port of the sequencing module, comprising a sequencing cartridge and sequencing flow cell.

FIG. 4F illustrates a side view of a sequencing module, comprising a sequencing cartridge and sequencing flow cell, that has received a nucleic acid sample prepared for sequencing.

FIG. 4G is a still photograph from a video of a robotic arm at rest position prior to receiving a signal from a software application.

FIG. 4H is a still photograph from a video of a robotic arm receiving an input from a system of the disclosure for grabbing a sequencing module, comprising a sequencing cartridge and sequencing flow cell, on a location and a software system communicating with the sequencing instrument to open the door to allow the inputting of the sequencing module.

FIG. 4I is a still photograph of a robotic arm inserting the sequencing module, comprising a sequencing cartridge and sequencing flow cell, into the sequencing instrument.

4J is a still photograph from a video of a robotic arm receiving an input from a system of the disclosure for complete loading of the sequencing module, comprising a sequencing cartridge and sequencing flow cell, onto the sequencer machine from a door on the sequencer machine that is configured to open to provide access to a cavity on the sequencer machine.

FIGS. 5A and 5B illustrates the robotic arm receiving an input from a software application of the disclosure for moving a reagent rack. FIG. 5C illustrates a robotic arm manipulating a reagent rack upon receiving an input from a software application of the disclosure.

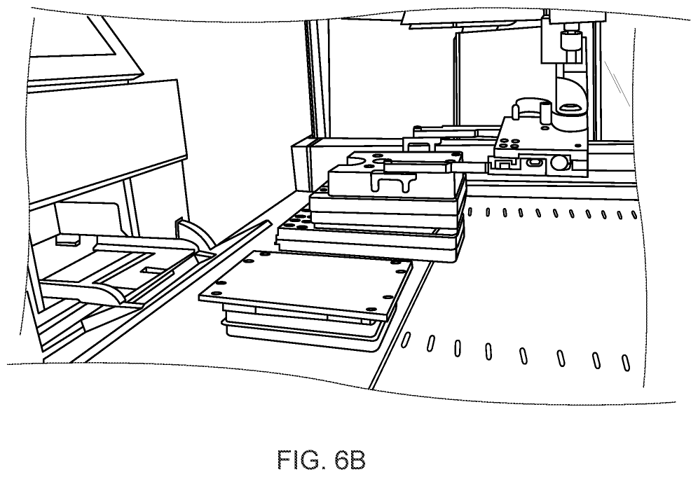

FIG. 6A illustrates the opening of a sequencer with an input from a software application of the disclosure.

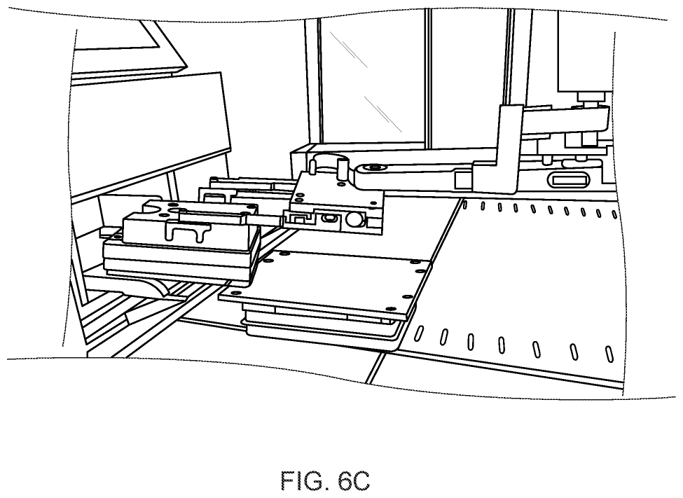

FIG. 6B-FIG. 6D collective illustrate the loading of a sequencer cartridge into a sequencer off-deck. The drawings are derived from still photographs taken of videos of a system of the disclosure actuating the fully automated preparation of nucleic acids from sample preparation to sequencing.

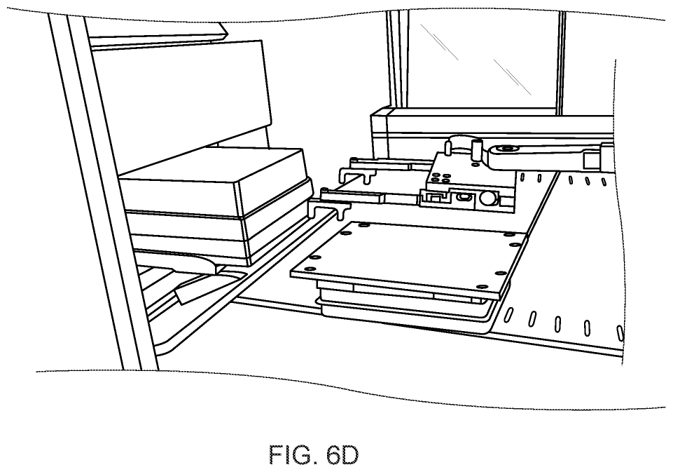

It should be understood that the drawings are not necessarily to scale, and that like reference numbers refer to like features.

### DETAILED DESCRIPTIONS

All of the functionalities described in connection with one embodiment of the methods, devices or instruments described herein are intended to be applicable to the additional embodiments of the methods, devices and instruments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

Over the past two decades, numerous bioanalytical techniques have been reported that use robotic liquid handlers to aliquot and/or extract target analytes from biofluids. One such automated bioanalytical extraction techniques uses solid phase extraction (SPE), robotic liquid-handling and sample-preparation workstations. Robotic liquid handlers have become the focus of portfolio execution via automated assay standardization and increased throughput. However, such systems remain limited to a defined number of processes. For instance, one exemplary system (e.g., Hamilton) has been automated for preparing a sample for nucleic acid sequence, yet, such system is not able to dynamically deviate from its pre-programmed instructions and provide variations depending on the genome to be prepared. Further, to date, there are no described systems that can automatically and robotically load a sequencing module, comprising a sequencing sequencer cartridge and sequencing flow cell, into a sequencer after a sample is prepared for nucleic acid sequencing.

The disclosure provides for the integration of an automation robotics workflow with a dynamic, integrated software system for fully automating sample preparation platforms from sample preparation to sequencing. As used herein, bioinformatics pipelines are used for data analysis of biologic parameters (e.g., genome size, expected number of reads based on sequencing size, expected depth of coverage from sequencing for a designated flow cell type) while software system is implemented in robotic control and automation of mechanical parts (e.g., actuate movement of a robotic arm). These enhancements are designed to perform more complex sample, biological, and labware manipulations, as well as integrate software programs and logistical database information, such as laboratory information management systems (LIMS). Other enhancements and customizations provided herein are aimed to increase the functionality of robotic arm movements, for integrating the loading of a sample into a sequencing module, comprising a sequencing cartridge and sequencing flow cell, and the loading of the sequencing module into a sequencer, when applicable. Further, various assay protocols used for biological or chemical research are concerned with performing a large number of defined and sequential reactions. The present disclosure developed and validated a complex, completely automated process for integrating all steps of nucleic acid extraction techniques (e.g., aliquoting, temperature control, vortexing, centrifugation, plate sealing) and functionally integrating them with the loading of such processed nucleic acids into a sequencing module, comprising a sequencing cartridge and sequencing flow cell, as well as the loading of the sequencing module into a sequencer.

Integration of an Automation Workflow with a Software System

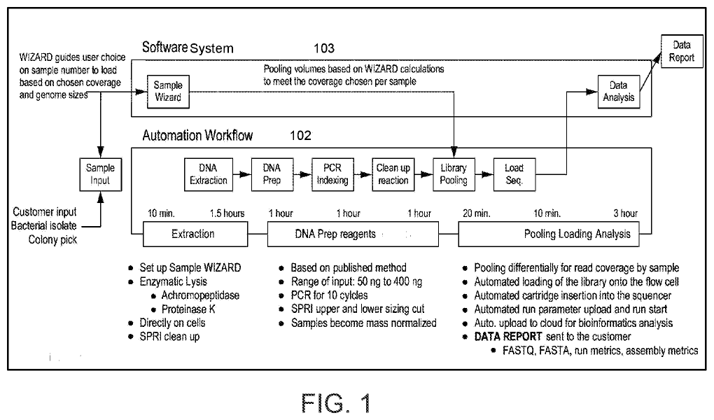

In many aspects, the disclosure provides a system, an apparatus, bioinformatics pipeline and an integrated software system comprising a plurality of software applications that support the full automation of a workflow for preparing nucleic acids for sequencing, and sequencing said prepared nucleic acids, exclusively using robotics (i.e., without human intervention). See FIG. 1. The apparatus can be part of a broader system functionally connected to a software application or computer program having executable code written therein for actuating a liquid handler and a robotic arm system for performing purification, target amplification, target pooling, indexing, attachment of sequencing adapters, attachment of proteins (e.g., attachment of polymerases, attachment of motor proteins for a pore sequencing workflow), and loading of nucleic acid samples prepared in such ways into a sequencing module, comprising a sequencing cartridge and sequencing flow cell, —optionally placed on a surface of a workstation or placed on a sequencer.

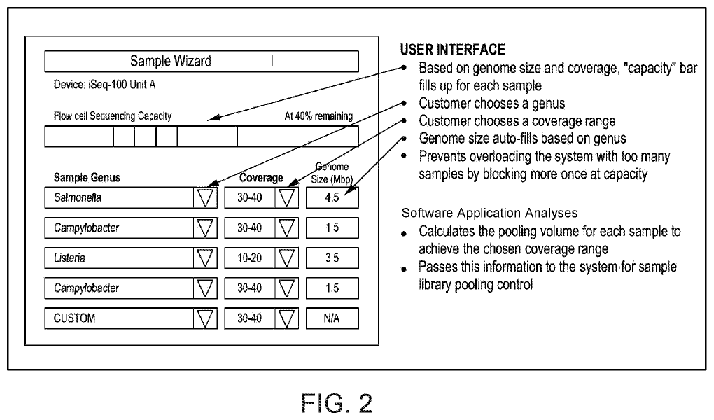

In many aspects, the integration of the software system facilitates the actuation of a fully automated system of the disclosure. For instance, FIG. 2 outlines biological parameter inputs and their deployment in a software application that, in conjunction with an integrated software system actuates the robotic components. In FIG. 2 the physical sequencing capacity of a flow cell (i.e., flow cell sequencing capacity), and a plurality of sizes of genomes associated with a variety of creatures, e.g., one or more of bacterial, viral, fungal, or mammalian genomes) was inputted as a parameter in a computer program custom developed by Applicants. An exemplary software application of such computer programs is depicted in FIG. 2. A user (e.g., a customer) then selected three exemplary genomes, in the illustrated case a user selected three unique genomes and one repeat: Salmonella (1), Campylobacter (2), Listeria (3). The software application, which was pre-programed with a genome size (Mbp) for a plurality of creatures (e.g., one or more of a virus, a bacterium, a mammalian, a fungi or an yeast genome), the flow cell sequencing capacity, was able to make a custom calculation of the pooling volume necessary for each sample to achieve the chosen coverage range. The software system was able to provide the calculated sample volume to the software application, which in turn used the sample volume calculated by the software application in the library pooling step of nucleic acid sample preparation (see, e.g., FIG. 1) for an overall schematic. The integrated software system can be components of a system that in turn actuates the fully automated preparation and sequencing of nucleic acid samples based on the input received from the integrated software application.

In some aspects, the disclosure provides an apparatus with the capability of supporting both macrofluidic and microfluidic reactions required for preparing a sample for sequencing with robotics, such apparatus can be, e.g., a sample preparation work-deck that actuates sample preparation based on input from software applications. The ability to execute macrofluidics and microfluidics reactions on the same robotic apparatus effectively support a variety of process that may be programmed into one or more workflows, including: centrifugation, filtration, magnetic bead capture, cell capture, electrokinetic processes that may aid in the purification of a sample; target amplification and indexing (optionally includes barcoding of samples); target pooling and purification; attachment of sequencing adapters; dilution of concentrated library into loading mix which may include beads and sequencing buffer; and loading into a sequencer. Such methods can be used in preparing libraries for next generation sequencing in a variety of platforms, including sequencing-by-synthesis, pore sequencing platforms, or single molecule sequencing platforms. As used herein, macrofluidic processors generally relate to macrofluidic devices for processing cell suspensions or clinical samples in the order of 100 μL to 10 mL (e.g., bodily fluids, swab specimens in preservation media). As used herein, microfluidic processors typically handle liquid between 1-100 μL volume. The present apparatus can be robotically controlled by a software application receiving instructions from the user through a graphical user interface (GUI), —but also compatible with a list of text commands—to allow a user to initiate a program for the complete preparation of a sample within the apparatus. Specifically, the computer program comprises commands for guiding liquid handling systems and robotic arms for moving samples around a work-deck (i.e., a workstation). Notably, the software application bridges the interactions between a robotic arm and, e.g., a liquid handling system with one or more pipet channels (e.g., eight pipet channels) and controls the workflow of the sample across a work-deck of the apparatus. Further, the software application bridges the interactions between a robotics system configured for grabbing and moving samples across a work-deck (i.e., a workstation) of the apparatus. In some configurations, the robotics arm may be able to actuate one or more of the functional steps handled by the microfluidics liquid handler (e.g., moving an input sample into a workstation).

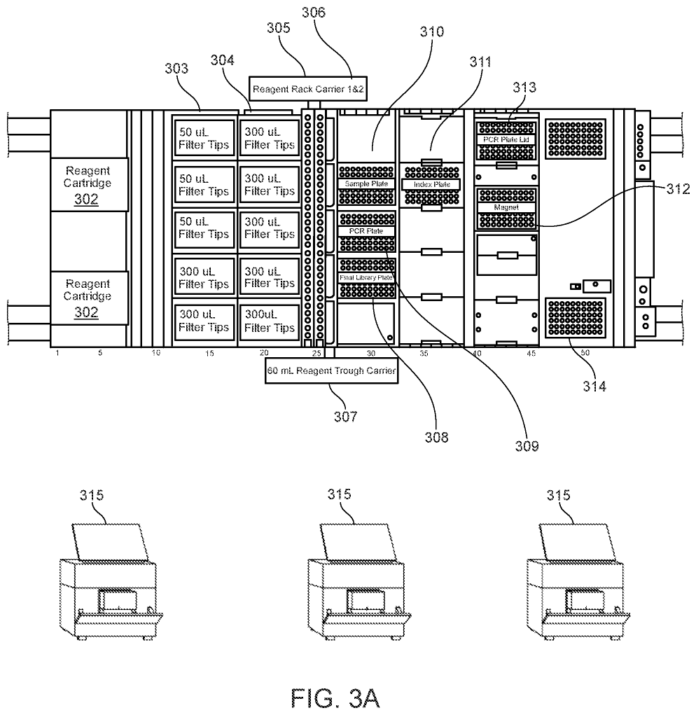

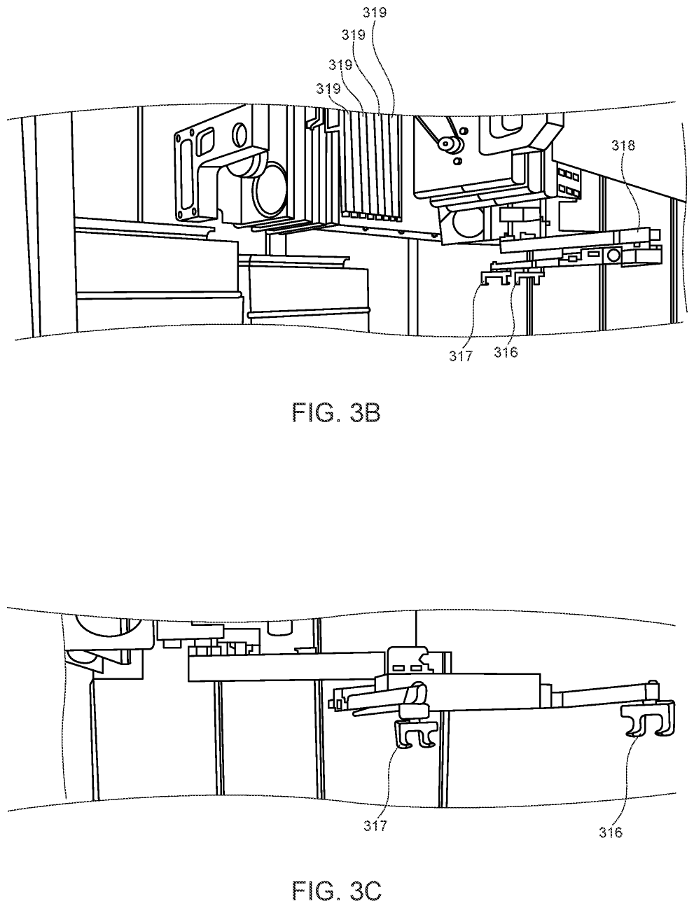

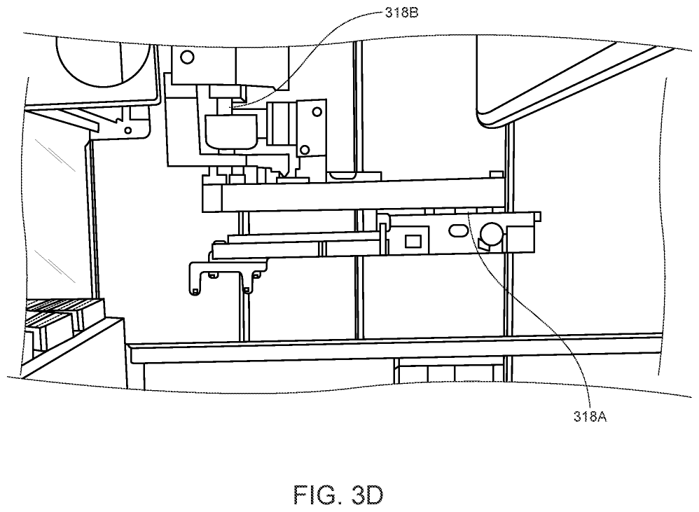

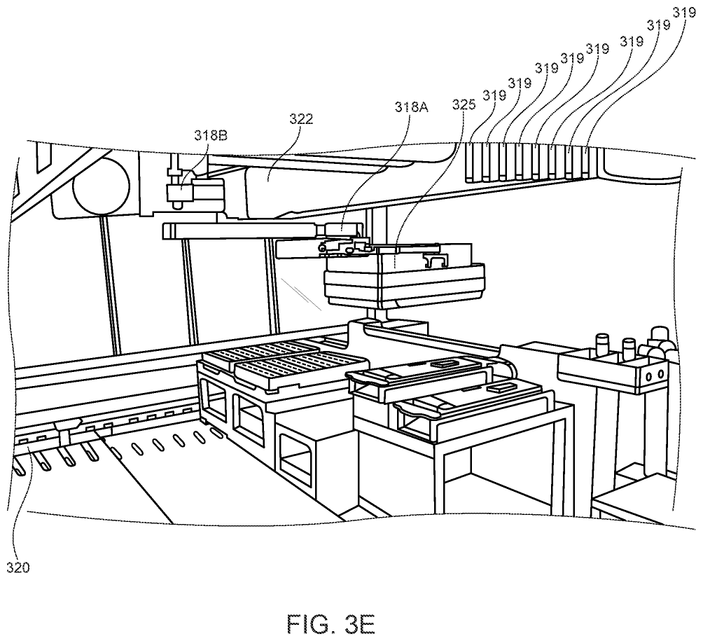

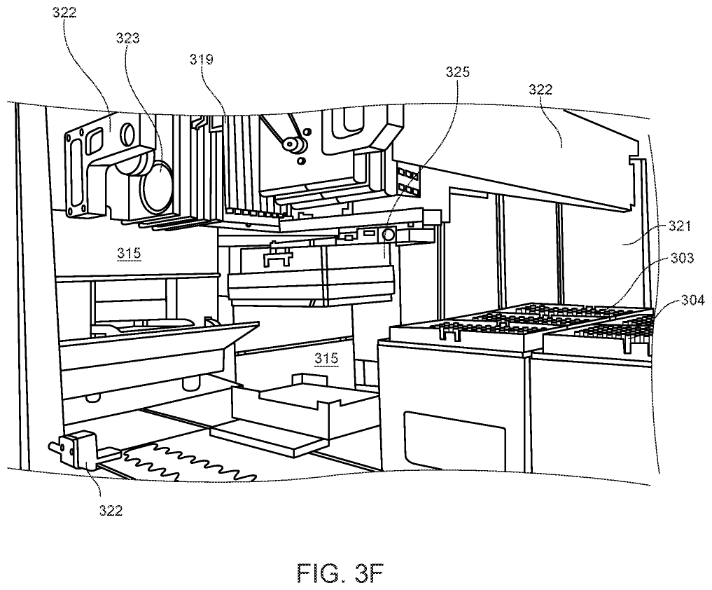

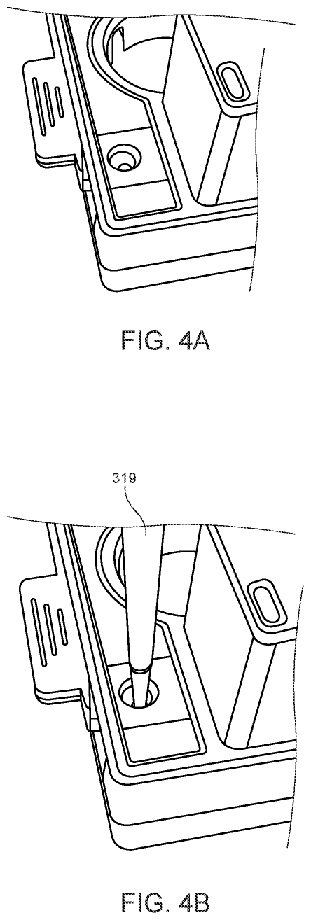

Generally, preparing a sample for sequencing often requires manipulating about 500 μL or more of a sample to single digit μL concentrations. In many cases, centrifugation, filtration, magnetic bead capture, cell capture, and electrokinetic methods are used to concentrate nucleic acids into smaller volumes. For example, some applications, such as the analysis of nucleic acids from microorganisms require first growing (e.g., enriching) said microorganisms by plating a sample on selective media plates, or growing the population of microorganisms in liquid selective media, and in some instances, performing further sub-culturing by transferred the culture to a new plate (i.e., sub-culturing) to obtain a purer isolate stock. Such processes can be performed for a variety of biological organisms, and can be actuated with a microfluidics liquid handling system. For an example of a suitable work-deck that can be integrated with bioinformatics pipelines and software system of the disclosure, see FIG. 3A. A system of the disclosure can, in some instances, receive a pooling volume calculated for each sample by a specific software application selected to achieve a chosen coverage area custom selected by a user. The integration of the software system and the bioinformatics pipelines can then be applied to actuate robotics movement to physically move the calculated pooling amounts into. Such calculation can prevent overloading the system with too many samples by blocking the addition of new samples once it is determined that the flow cell would be at capacity at the end of the run.

Apparatuses of the disclosure are configured to seamlessly connect macrofluidic and microfluidic workflows by applying robotically controlled liquid handling systems for moving samples into and out of different chambers, e.g., chambers functionally connected to a vacuum manifold or chambers functionally connected to a thermal cycler (which can be placed on deck or off deck). In many aspects, the disclosure provides an apparatus that may have a plurality of chambers as necessary to support the processing of nucleic acids for a particular sequencing application. Such chambers can be placed on a work-deck. In some cases, the plurality of chambers are housed within a lodging and in other cases they are stand-alone. In some cases, the plurality of chambers includes a sample chamber, a cell culture chamber, a heater, a thermal cycler, a vacuum manifold, a purification chamber (e.g., a magnetic deck and associated components), a waster container, a centrifuge, a reagents container, a consumables container (e.g., pipette tips). Depending on the application, the disclosure may provide apparatuses having two or more, three or more, four or more, or five or more sample chambers, cell culture chambers, heaters, thermal cyclers, purification chambers, waste containers, centrifuges, reagent containers, or consumables containers (e.g. pipette tips). Each chamber may be placed on its own on a surface of a workstation. Alternatively, each chamber may be placed in one or more racks atop the workstation. A rack may provide support by physically restraining the chamber to a particular location. Placement of the chambers on a rack may facilitate the programming of a computer system that actuates the automation of the process for extracting nucleic acids, target amplification and indexing, target pooling and purification, and attachment of sequencing adapters.

An apparatus of the disclosure can be actuated by a cloud-based software system or localized computer-program, e.g., comprises a plurality of software applications networked together via a plurality of APIs or receiving commands from users through a graphical user interface (GUI) or a series of text commands from a software application with at least one liquid handler, e.g. a Hamilton STAR robotic liquid handler as both a macrofluidics handler and a microfluidics handler. A software system comprises a plurality of software applications networked together via a plurality of APIs. Bioinformatics pipelines are used for data analysis, while software system is implemented in robotic control and automation of mechanical parts, and to calculate metrics used in measuring the necessary quantities for the capacitor flow cell. The software application can be programmed using proprietary in-house developed cloud-based platform Java Spring Framework and combined with all associated study and extraction specific variables and information (including a LIMS worklist) into a database file that can be imported into the liquid-handling workstation's method. The software application can be programmed to sequentially perform the microfluidics and microfluidics steps of the protocol. The Hamilton Microlab STAR's (Hamilton Robotics, Reno, NV, USA) liquid-handling method, for example, can be programmed within a specific software application, or another suitable software or version, to execute all sample-preparation commands for all microfluidics modules. Microfluidic modules based on electrowetting or pressure-driven microfluidics or other electrokinetic mechanisms for executing certain parts of the sequencing workflow can be integrated into the Hamilton liquid handling platform. In such cases, the controller software application for those modules can be programmed to execute all sample-preparation commands for all microfluidics modules. Such a workflow allows full integration of macrofluidics to microfluidics protocols within one apparatus, for example, within a series of chambers placed atop a work-deck of the apparatus and available for interacting with the macrofluidics liquid handler and the microfluidics liquid handler. Further, such a workflow supports the actuation of a robotic system, such as, e.g., a robotic system having a robotic arm (318) having a left effector (317) and a right effector (316) connected to a rotary axis (collectively, 318A and 318B) for actuating one or more programmed movements of the robotic arm, see FIGS. 3B-3D. In such configurations, the robotic arm receives an, preferably automated, input from the software application for moving said robotic arm towards a sequencing module, comprising a sequencing cartridge and sequencing flow cell, on a location, extending the left effector and the right effector of the robotic arm around the sequencing module on the location and subsequently contracting said effectors around the sequencing module, thereby holding the sequencing module with the robotic arm. See, e.g., FIG. 3E. In these configurations, the software application is programmed for moving the sequencing module that is held by the robotic arm into a sequencer machine thereby loading the sequencing module. See, e.g., FIGS. 3E and 3F. In many aspects, the rotary axis for the robotic system is configured for rotating 360°, which allows the left effector and the right effector to rotate to suitable position for grabbing, e.g., the sequencing module, a rack, or a container within a rack in a location housing these objects. In preferred instances, the left effector and the right effector are symmetrical, but alternative embodiments with asymmetrical effectors are also contemplated herein. In the configuration deployed in the instant experiments, a software application instructs the movement of the robotic arm, which is physically attached to the work deck by the movable support (322), which can be connected to side support, by “sliding” it backwards and forwards over the work-deck on a movable belt. An end of a movable belt is illustrated to underscore that the robotic arm utilized robotic components (e.g., 323, 322) to extend the lifting of the sequencing cartridge beyond the surface of the work-deck (320), and completely into the sequencer. FIG. 3F also depicts that the software system can automatically open the door of the sequencer. A system of the disclosure can optionally be encased by a surrounding that is not part of the system (321), or it can be in an open environment.

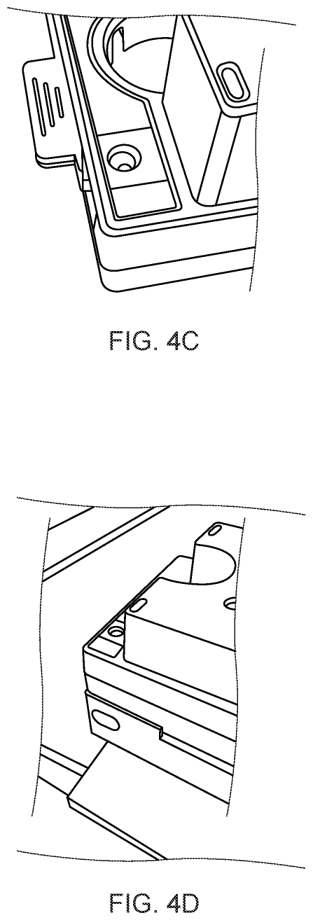

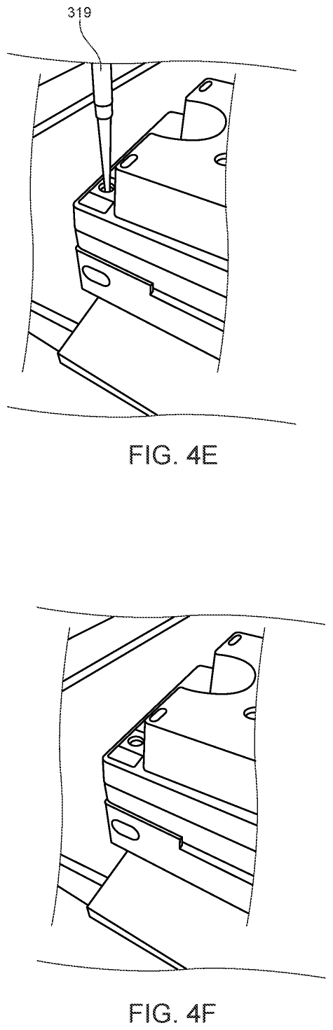

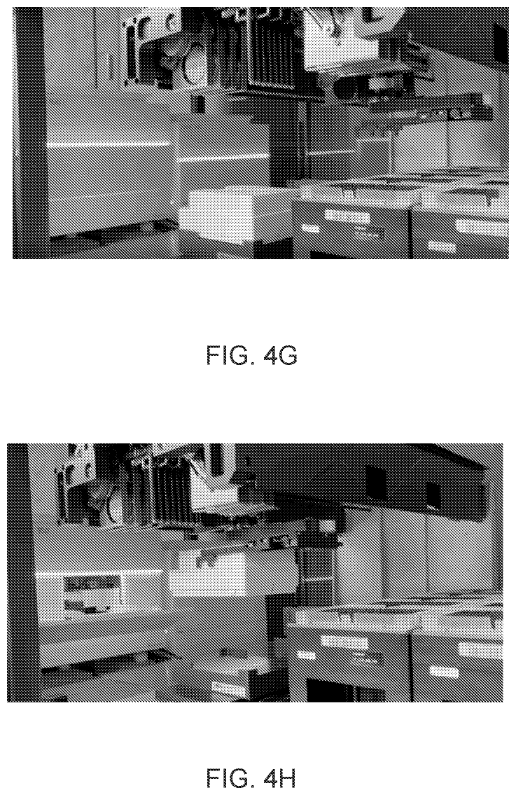

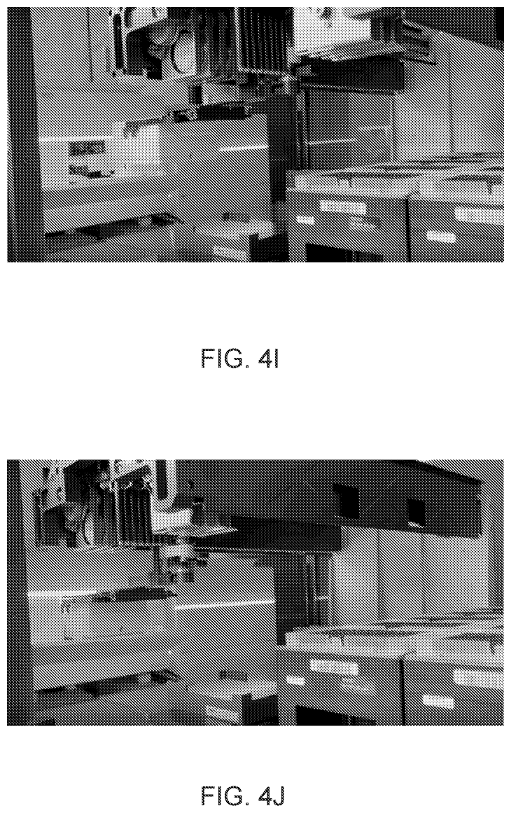

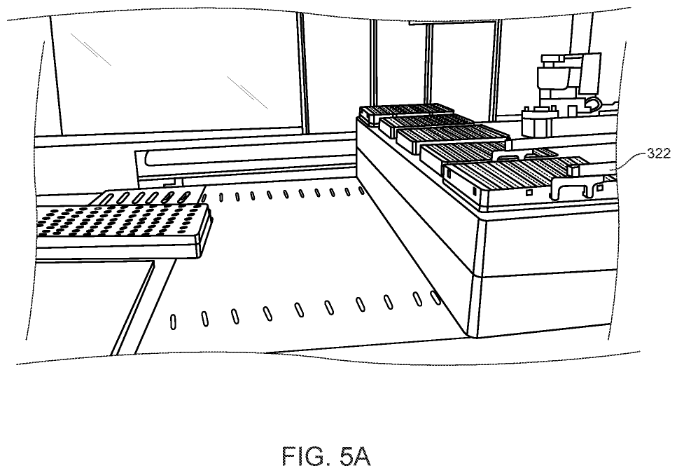

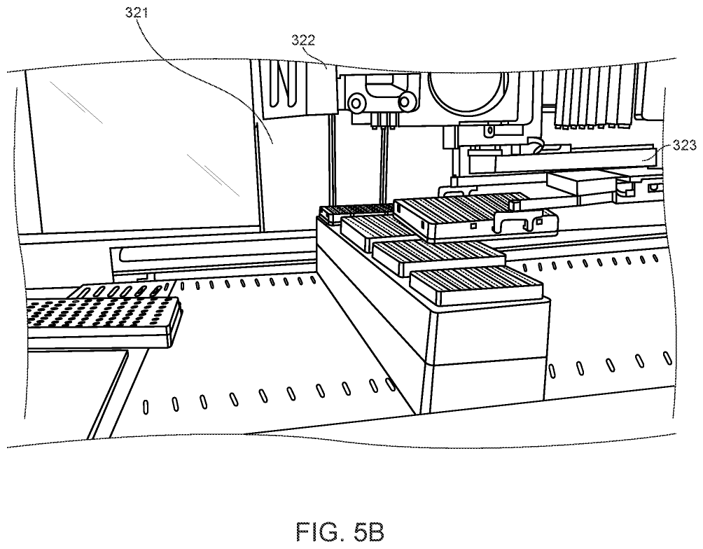

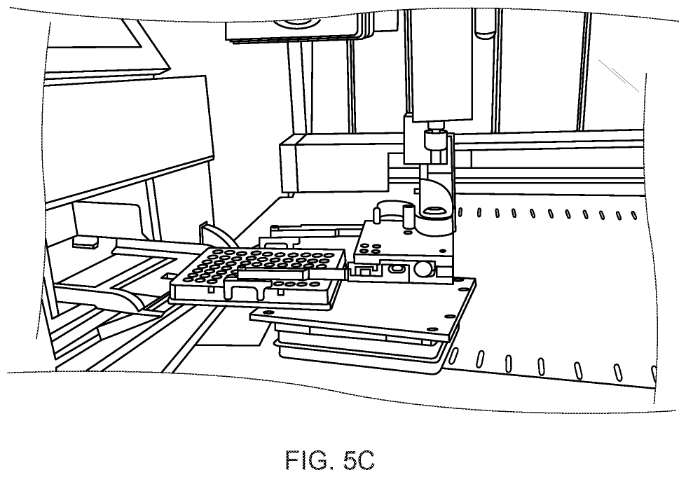

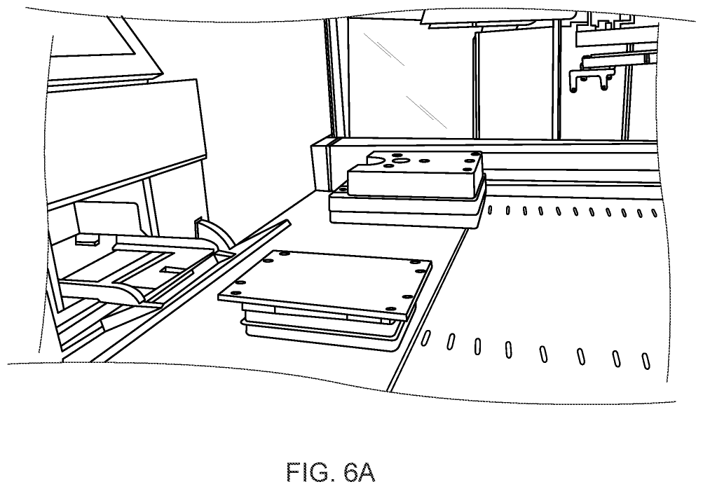

To accommodate the weight of a sequencing cartridge—and flow cells—designed to be compatible with different sequencers, e.g., U.S. D782,690, U.S. RE48,219, U.S. RE48,561, U.S. Pat. Nos. 8,914,241, 8,921,073, 11,117,130, 9,365,898, U.S. RE48,219, or U.S. RE48,561, the left effector and the right effector can be configured for lifting at least 1 pound, at least 2 pounds, at least 5 pounds, at least 10 pounds, or a suitable weight for the sequencing cartridges, flow cells, racks, and containers used in steps of the automated process for purifying (extract and concentrate) to, e.g., single digit μL concentration a sample input to one or more chambers optionally placed within racks on a workstation. See FIG. 3. Depending on the configuration of the system, either the liquid handler or the robotic system having a left and a right actuator is used to move samples across each step of the process. In some configurations, the sequencer machine is configured for performing sequencing-by-synthesis. The disclosure contemplates that the flow cell sequencing capacity of one or more flow cells compatible with the above referenced sequencers, and other suitable flow cells, can be inputted into a software application described herein. This information can, in some instances, be used to inform the loading of a flow cell. See FIGS. 4A to 4F. FIGS. 4A-4F depict an exemplary loading of sequencing module, comprising a sequencing cartridge and sequencing flow cell, in the instant case by piercing the loading port of the sequencing cartridge with a pipet attached to a liquid handling system of the disclosure. The instant disclosure also contemplates that automated loading can be performed as described in U.S. Pat. Nos. 11,282,587; 10,597,714.

In some configurations, the sequencer machine is configured for receiving one or more inputs directly from the software system. For instance, power switches on a sequencer used with a system of the disclosure can be automatically actuated (i.e., turned on and off) in a variety of ways. In one configuration, the software application actuating the automated process sends an electronic input to the sequencer instructing it to turn on. In another configuration, the robotic arm system (i.e., the system having the left actuator and the right actuator) is used to robotically (e.g., physically) power the sequencer on. The robotic arm is capable of moving 360° above and beyond the surface of the workstation. In some configurations the sequencer machine automatically starts a sequencing run upon loading of the sequencer cartridge.

In some configurations a system of the disclosure is functionally connected to an apparatus capable of actuating a liquid handling system, which can comprise one or more pipet channels (319). The liquid handling system can be functionally connected to a multi-module workstation. For instance, as described above, the controller software for those modules can be programmed within a specific software application to execute all sample-preparation commands for all microfluidics modules. A variety of liquid handling systems can be robotically programed to actuate commands from an apparatus of the disclosure. In some instances, a liquid handling system of the disclosure is a micro-pipetting system that uses a positive displacement pipetting system. Positive displacement pipetting is often based on direct contact of the piston with the liquid. The aspirated liquid amount depends on the dimensions of the cylinder or capillary and the movement distance of the piston. Positive displacement pipettes generally provide numerous advantages. For example, cross contamination between different samples aspirated by the same pipette (e.g., via aerosol) is significantly reduced. Moreover, positive displacement pipettes often provide significantly higher accuracy and precision that is typically not achieved with air-interface pipettes, especially where high-vapor pressure liquids, detergent containing fluids, and/or volatile solvents are aspirated. In positive displacement pipettes the tips contain both the cylinder/capillary and the piston. Non-disposable pipette tips (e.g., Teflon coated tips) for positive displacement pipettes may be used in robotic pipettors and may be washed between uses to remove potential cross contamination. In many instances, a liquid pressure can drive positive displacement pipetting system. An instrument of the disclosure may have more than one liquid handling system, for example, a macrofluidics liquid handling system for handling larger volumes (e.g., cell culture samples of 50 μL to 1,000 mL, from 50 μL to 2,000 mL, 500 μL to 5,000 mL, 500 μL to 10,000 mL, or another suitable volume range) and a microfluidics liquid handling system for handling nanoliter volumes (e.g., enzyme volumes and loading into an input port on a flow cell.

A plurality of macrofluidics (e.g., system handling 5 mLs or above) and microfluidic liquid handling systems can be easily configured for processing samples from one workstation (e.g., one module to another). For instance, one liquid handling system may be configured for moving from 500 mL of liquid to 25 mL of liquid, from 500 mL of liquid to 20 mL of liquid, from 500 mL of liquid to 15 mL of liquid, from 500 mL of liquid to 12 mL of liquid, from 500 mL of liquid to 10 mL of liquid, from 500 mL of liquid to 5 mL of liquid, or from 500 mL of liquid to 2 mL of liquid; while another liquid handling system is configured for moving microliter or sub-microliter volumes of liquids as described elsewhere herein. In such instances, the apparatus is configured to provide a macrofluidic to microfluidic interface that can be used, for example, to use a first liquid handling system to move live cells grown in media to a centrifuge or another suitable module to be concentrated. After concentration, the apparatus may be configured to move a cell pellet that is resuspend in a significantly smaller volume of liquid (a volume amount that is less than 2 mL, less than 1 mL, less than 500 μL, or another suitable amount) into one or more of the sample preparation chambers described herein. In many such instances, one or more robotic macrofluidic devices are configured for aspirating fluids from the sample processor and into the microfluidic sample preparation cartridge. In other instances, one or more robotic macrofluidic devices are configured for aspirating fluids from the sample processor and into the microfluidic sample preparation cartridge and from the sample preparation cartridge into the flow cell.

As described here, microfluidic sample preparation cartridges may use pressure-driven microfluidics, droplet microfluidics, digital microfluidics, or centrifugal microfluidics for moving the sample through the one or more conduits in the sample preparation cartridge(s). See FIG. 3 for an exemplary work-deck (workstation) configuration of the apparatus. FIG. 1 provides an overall overview of the integration of an automation workflow with a software system.

In other instances, a microfluidics liquid handling system of the disclosure can be an air displacement pipetting system. Air displacement pipetting is highly accurate for standard pipetting applications. However, operating conditions such as temperature and atmospheric pressure, as well as the specific gravity and viscosity of the solution, may affect the performance of air displacement pipettes. Alternatively, a positive displacement pipetting system can be an ultrasound transducer pipetting system, see e.g., US20050124059A1. In other cases, a positive displacement pipetting system can be a miniaturized pipetting system such as a capillary driven pipetting system.

In some instances, an apparatus for automating a workflow from nucleic acid sample preparation to nucleic acid sequencing, can comprise: a liquid handling system having one or more pipetting channels configured for transferring a plurality of liquids into and out of a plurality of individual wells, where the pipetting channels is functionally connected to a robotic pipetting arm for automatically moving the pipetting channels within the apparatus; at least one chamber for holding a plurality of nucleic acids; a thermal cycler having a multi-well plate unit mounted therein configured for controlling the temperature of the multi-well plate unit; a vacuum manifold having a multi-well plate having a plurality of columns at the bottom of each well unit mounted therein; and a flow cell functionally connected to a sequencer device; whereby the apparatus is configured for automatically using the robotic pipetting arm for moving the pipetting channels within the apparatus, whereby the pipetting channels is configured for automating the workflow from nucleic acid sample preparation to nucleic acid sequencing by: moving the plurality of nucleic acids from the at least one chamber into the multi-well plate unit mounted on the thermal cycler for generating a plurality of amplified target nucleic acids; moving the plurality of nucleic acids from the multi-well plate unit mounted on the thermal cycler into the multi-well plate having the plurality of columns for removing a set of non-amplified nucleic acids from the plurality of amplified target regions via vacuum by the vacuum manifold; moving the plurality of amplified target nucleic acids from the multi-well plate having the plurality of columns into the flow cell functionally connected to the sequencer, whereby the apparatus is configured to instruct the sequencer device to perform the sequencing reaction thus automating the workflow from nucleic acid sample preparation to nucleic acid sequencing. This apparatus can control a first liquid handling system for handling macrofluidic volumes and a second liquid handling system for handling microfluidic volumes.

In other instances, an apparatus for automating a workflow from nucleic acid sample preparation to nucleic acid sequencing can comprise a liquid handling system having one or more pipetting channels configured for transferring a plurality of liquids into and out of a plurality of individual wells, where the pipetting channel is functionally connected to a robotic pipetting arm for automatically moving the pipetting channel within the apparatus; one or many chambers for holding a plurality of nucleic acids; a thermal cycler having a multi-well plate unit mounted therein configured for controlling the temperature of the multi-well plate unit; a magnet array having a multi-well plate unit mounted therein; and a flow cell functionally connected to a sequencer device; whereby the apparatus is configured for automatically using the robotic pipetting arm for moving the pipetting channel within the apparatus, whereby the pipetting channel is configured for automating the workflow from nucleic acid sample preparation to nucleic acid sequencing by: moving the plurality of nucleic acids from one of the many chambers into the multi-well plate unit mounted on the thermal cycler for generating a plurality of amplified target nucleic acids; moving the plurality of amplified target nucleic acids from the multi-well plate unit mounted onto the thermal cycler into the multi-well plate mounted onto the magnet array for separating a set of non-amplified nucleic acids from the plurality of amplified target regions via bead-capture by magnetic beads within the multi-well plate mounted onto the magnet array; moving the plurality of amplified target nucleic acids from the multi-well plate into the flow cell functionally connected to the sequencer, whereby the apparatus is configured to instruct the sequencer device to perform the sequencing reaction thus automating the workflow from nucleic acid sample preparation to nucleic acid sequencing. This apparatus can control a first liquid handling system for handling volumes and a second liquid handling system for handling microfluidic volumes.

An apparatus of the disclosure 300 may have a plurality of modules in the multi-module workstation. The disclosure contemplates embodiments where a sample is prepared for sequencing within such modules without any human intervention. The plurality of modules may have chambers supported by a housing, such as a rack for lodging a sample chamber. In some instances, the workstation deck has a sample preparation module (not shown in FIG. 3) (e.g., a sample module having a sample chamber) contained in a rack for holding and storing samples. The sample module may be placed off deck or it may be placed on the work-deck of an apparatus. The sample module may be temperature controlled and may, for example, hold samples at 4° C. for a defined period-of-time, or it may hold samples at room temperature. The sample may warm up samples to suitable temperatures for cell growth, such as 37° C. The microfluidic controller may be pre-programed to automatically instruct the robotic arms movements for using a liquid handling systems (i.e., a macro-pipetting system) for moving a sample from a sample module into a DNA extraction module. The work-deck may be divided into modules, meaning programmed sections of a nucleic acid preparation protocol, e.g., DNA extraction, DNA preparation, PCR indexing, clean up reaction, library pooling reaction, and the loading of a sample into a sequencing cell that are written into a computer program or software application actuating the robotics within the apparatus.

Within each module of the workstation, for example, a system may comprise a plurality of multi-well plates for receiving samples. Either a liquid handler of the disclosure of the robotic arm may be configured to move a nucleic acid sample to one or more modules of the workstation. The workstation may comprise a plurality of racks. The plurality of racks may include chambers for holding reagents for extracting a nucleic acid from a cell, e.g., reagents for cell lysis. The plurality of racks may include chambers for holding reagents for fragmentation of a nucleic acid. The plurality of racks may include chambers for holding reagents for end repair of a nucleic acid. The plurality of racks may include chambers for holding reagents for fragmentation of a nucleic acid. The plurality of racks may include chambers for holding reagents for reverse transcription of one or more nucleic acids. The plurality of racks may include chambers for holding reagents for amplification of one or more nucleic acids. The plurality of racks may include racks for holding reagents for adding a sequencing adaptor to one or more nucleic acids. A liquid handler may be actuated by the software application to draw in (e.g., aspirate) samples from one or more of these chambers and draw out the samples after a chemical reaction is deemed to be complete. In some configurations a robotic arm may be programmed to actuate the movement of, e.g., a sample contained in a chamber into and out of a magnet plate, a vacuum manifold, a thermal cycler present atop the workstation. In other configurations a liquid handler may be programmed to actuate the movement of, e.g., a sample contained in a chamber into and out of a magnet plate, a vacuum manifold, a thermal cycler present atop the workstation.

The work-deck 300 may have a plurality of modules supported by a housing, such as a housing for lodging a thermocycler, a purification chamber, a magnetic bead separator, and a flow cell (i.e., cartridge) that can be functionally connected to a sequencer cartridge. In many instances, automation of the workflow from sample preparation to nucleic acid sequencing requires the steps of target amplification and indexing, target pooling and amplification, and attachment of sequencing adapters, all of which can be automatically performed. To facilitate the workflow, the liquid handling system can be configured for automatically moving a sample from the cell culture shaker into and out of the thermal cycler, into and out of the purification chamber, or into the flow cell. The robotic arm can be configured to move the liquid handling system for automatically accessing reagents in one or more reagent modules within the apparatus workstation deck. The robotic arm may be configured to move the liquid handling system for automatically loading and retrieving samples from a centrifuge. The housing may optionally lodge an absorption and fluorescence spectroscope for measuring the concentration of cells or nucleic acid in the input sample or at any immediate step of the workflow respectively. In some configurations the workstation is an open workstation.

In some instances, the apparatus workstation further lodges a centrifuge. The centrifuge can, for example, be used to spin down and concentrate the cells in the sample input. The robotic arm can be configured for automatically moving a sample into and out of the thermal cycler, into and out of the purification chamber, into and out of the flow cell, or into and out of the centrifuge, or into the flow cell.

In some instances, the apparatus workstation further lodges a waste container. The waste container may receive all liquid discards from the liquid handling system. In some instances, an additional module may be present to support washes of a macrofluidic or microfluidic liquid handling system. The macro- or micro-fluidic controller may be configured to move the robotic arm functionally connected to the liquid handling system for a system wash with a liquid present in the additional module.

In some instances, the apparatus can actuate a liquid handler to load a nucleic acid sample prepared for sequencing by piercing the cover over the loading port of a sequencing cartridge. FIG. 4A illustrates a front view of a sealed loading port of a sequencing cartridge and FIG. 4B illustrates the piercing of the cover over the loading port of a sequencing cartridge by the liquid handler. FIG. 4C illustrates the loading port of a sequencing cartridge was pierced and loaded with nucleic acids prepared for sequencing. FIGS. 4D-4F. illustrates a side view of that same process.

Integration of Software Applications into a Software System for Actuation of One or More Steps in Automation of a Workflow for Nucleic Acid Sequencing

In some aspects, the disclosure provides a computer program product comprising instructions which, when the program is executed by a computer actuate the integration of an automation (physical) workflow with a software system for the automated preparation (see FIG. 1, FIG. 2, FIGS. 3A-3F, and FIGS. 4A-4F. In some aspects, the disclosed provides a computer program or software application product comprising instructions which, when the program is executed by a computer, cause the computer to instruct a robotic system insert a sequencing module, comprising a sequencing cartridge and sequencing flow cell, into a sequencer.

In many instances, systems, platforms, software, networks, and methods described herein include a digital processing device, or use of the same. In further embodiments, the digital processing device includes one or more hardware central processing units (CPUs), i.e., processors that carry out the device's functions, such as receiving an input from a user to provide an experimental planning wizard, or a capacity meter, that can help plan a nucleic acid sequencing experiment a priori for a mixed set of samples (pathogen types, coverage desired, genome size, pathogen gram stain type, etc.) and dynamically actuate a liquid handling protocol based on the input received. The localized computer or cloud-based software system, e.g., a plurality of software applications, disclosed herein or a computer system used in the analyses of a set of short (1×75 to 2×300) nucleic acid sequencing read results, and/or long nucleic acid sequencing read results (in the order of kb) results, can share the results with a third-party from any other facility, such as a hospital, a clinic, or another facility. In still further embodiments, the digital processing device further comprises an operating system configured to perform executable instructions. In some embodiments, the digital processing device is optionally connected to a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device. In other embodiments, the digital processing device could be deployed on premise or remotely deployed in the cloud.

In many embodiments, a software application comprising a plurality of bioinformatics pipelines is configured to combine short and long read assemblies to construct a hybrid de novo assembly. For instance, the required sequencing capacity can depend on the size of one or more target regions of interest, the types of variant and the disease model being analyzed. A coverage depth needed for a particular application can then be selected by a user (e.g., customer) on the described software application. The breadth of coverage (the percentage of target bases that have been sequenced for a given number of times) can thus be adjusted based on a selection made by a user.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art. In many aspects, the disclosure contemplates any suitable digital processing device that can be programmed for integration of an automation workflow with the software system. In some instances, the disclosure contemplates improvements of digital processing devices that have integrated multiple assay extraction platforms (e.g., Hamilton®) using robotic liquid handling that support the addition of new robotic handling systems, new robotic arms, and new functionalities (e.g., loading of a sample or a plurality of samples onto a flow cell).

In some embodiments, a digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeB SD, OpenBSD, NetBSD, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, a digital processing device includes a storage and/or memory device. The storage and/or memory device is one or more physical apparatuses used to store data or programs on a temporary or permanent basis. In some embodiments, the device has volatile memory and requires power to maintain stored information. In some embodiments, the device has non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing-based storage. In further embodiments, the storage and/or memory device is a combination of devices such as those disclosed herein.

In some embodiments, a digital processing device includes a display. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, a digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera to capture motion or visual input. In still further embodiments, the input device is a combination of devices such as those disclosed herein. In many instances, the input device is where a user first makes a selection based on genome size of an organism and desired coverage. The input device then displays a “capacity” bar, which fills up for each selected flow cell. A user chooses a genus and a coverage range. Based on the genus selection, the genome size auto-fills and is displayed in the input device. See, e.g., FIG. 2. Such calculations notably prevents overloading the selected flow cell with too many samples by blocking more once at capacity.

In some embodiments, a digital processing device includes a digital camera. In some embodiments, a digital camera captures digital images. Such digital camera may capture digital images of the automation workflow, e.g., in some instances the loading of a flow cell of FIGS. 4A-4D may be captured with a digital camera. In some embodiments, the digital camera is an autofocus camera. In some embodiments, a digital camera is a charge-coupled device (CCD) camera. In further embodiments, a digital camera is a CCD video camera. In other embodiments, a digital camera is a complementary metal-oxide-semiconductor (CMOS) camera. In some embodiments, a digital camera captures still images. In other embodiments, a digital camera captures video images. In various embodiments, suitable digital cameras include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and higher megapixel cameras, including increments therein. In some embodiments, a digital camera is a standard definition camera. In other embodiments, a digital camera is an HD video camera. In further embodiments, an HD video camera captures images with at least about 1280×about 720 pixels or at least about 1920×about 1080 pixels. In some embodiments, a digital camera captures color digital images. In other embodiments, a digital camera captures grayscale digital images. In various embodiments, digital images are stored in any suitable digital image format. Suitable digital image formats include, by way of non-limiting examples, Joint Photographic Experts Group (JPEG), JPEG 2000, Exchangeable image file format (EXIF), Tagged Image File Format (TIFF), RAW, Portable Network Graphics (PNG), Graphics Interchange Format (GIF), Windows® bitmap (BMP), portable pixmap (PPM), portable graymap (PGM), portable bitmap file format (PBM), and WebP. In various embodiments, digital images are stored in any suitable digital video format. Suitable digital video formats include, by way of non-limiting examples, AVI, MPEG, Apple® QuickTime®, MP4, AVCHD®, Windows Media®, DivX™, Flash Video, Ogg Theora, WebM, and RealMedia. In specific instances, such images can become part of the subjects' medical record.

Non-Transitory Computer Readable Storage Medium

In many aspects, the systems, processes, software, networks, and methods that actuate the automation workflow, the software system, and the integration of both disclosed herein include one or more non-transitory computer readable storage media encoded with a program including instructions executable by the operating system of an optionally networked digital processing device. For instance, in some aspects, the actuation requires creating data files associated with a plurality of user selections for a particular nucleic acid sequencing experiment. In preferred embodiments, the user selection drives the calculation of the pooling volume for each sample to achieve the chosen coverage range. The determination of the pooling volumes based on a specific software application, e.g., (Wizard) calculations to meet the coverage chosen per sample is the physical point of integration where software system 103 provides an input to automation workflow 102. These calculations are typically conducted prior to the start of the sequencing experiment.

Once such calculations are executed, the software application, e.g., sample Wizard, may instruct, depending on the configuration of the system deployed, either a liquid handling system or a robotic arm, to add a sample having nucleic acids therein into a chamber for direct enzymatic lysis (e.g., achromopeptidase, proteinase K), chemical lysis or physical lysis. Subsequently, the software application actuates either the liquid handling system or the robotic arm, to SPRI clean up. The aforementioned DNA extraction can take from 10 min to 1.5 hours. Once the DNA has been extracted, the software application actuates the liquid handling system to move the extracted nucleic acids through a process for DNA preparation. A DNA preparation protocol can be executed by the software application based on the sequencing method selected. For instance, sequencing-by-synthesis protocols executes herein have been executed with a range of input from 50 ng, 60 ng, 70 ng, 80 ng, 90 ng, 100 ng, 110 ng, 120 ng, 130 ng, 140 ng, 150 ng, 160 ng, 170 ng, 180 ng, 190 ng, 200 ng, 210 ng, 220 ng, 230 ng, 240 ng, 250 ng, 260 ng, 270 ng, 280 ng, 290 ng, 300 ng, 310 ng, 320 ng, 330 ng, 340 ng, 350 ng, 360 ng, 370 ng, 380 ng, 390 ng, to 400 ng of input purified nucleic acid, PCR for 5-15 cycles*, SPRI clean up and sample auto-normalization. At this junction, the pooling volume calculations from the software system 103 informed the library pooling of the automation workflow 102. By piercing the loading port of the sequencing cartridge, the actuated liquid handler automated a loading of the prepared nucleic acid sequencing library into the sequencing flow cell via the sequencing cartridge, however the disclosure contemplates alternatives to piercing of any other components, e.g., leaving a flow cell, e.g., Oxford Nanopore® flow cell already open atop of a workstation. Subsequently, the robotic arm automated the sequencing module, comprising a sequencing cartridge and sequencing flow cell, insertion into the sequencer. In some configuration the disclosed system automated run parameter upload and run starts. The non-transitory computer storage medium can store data files associated with all of the measurements described herein.

In many embodiments, a computer software of the disclosure comprises an operating system configured to perform executable instructions, including combining short range and long range sequencing reads. For instance, an exemplary sequencer, the iSeq™ is capable of short reads only (150 bp times 2 for total short reads) and the flow cell for the iSeq is limited for short read capacity. Nonetheless, longer sequencing results can be obtained with different sequencing technology, e.g., ONT. Both sequences can be part of a methods of the disclosure, see FIG. 3, for example. In some aspects, a bioinformatics pipeline of the disclosure is configured to perform a hybrid analysis of long and short range sequences as described herein.

Further the systems, apparatus, liquid handlers, robotic arms, processes, software, networks, and methods that provide the integration of an automation workflow with a software system disclosed herein include one or more non-transitory computer readable storage media encoded with a program including instructions executable by the operating system of an optionally networked digital processing device configured to create data files associated with a plurality of sequencing reads (and associated sequencing read measurements) measurements from a short sequencing read, a long sequencing read, or both. In preferred embodiments, the data is further structured by a matrix that generates hybrid assemblies (short, long, and both) into combinations of short sequencing reads for selected genome 1, long sequencing reads for selected genome 1, (optionally) short sequencing reads for selected genome 2, long sequencing reads for selected genome 2, and potential combinations of the user selections. The generation of such hybrid assemblies in an automated fashion support the ability of a system of the disclosure to sort short nucleic acid sequencing reads and long nucleic acid sequencing reads from one sample in the same platform with the same automated fashion and provide a data report to a customer comprising FASTQ, FASTA, run metrics, and assembly metrics. The non-transitory computer storage medium can store data files associated with all measurements taken during the integration of an automation workflow with a software system described herein.

In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media. Such computer readable storage medium is also suitable for storing the set of data contemplated by the disclosure.

Computer Program

In some embodiments, the systems, platforms, software, networks, and methods disclosed herein include at least one computer program. A localized computer program or cloud-based software system includes a sequence of instructions, executable in the digital processing device's CPU, written to perform a specified task. In light of the disclosure provided herein, those of skill in the art will recognize that a localized computer program or cloud-based software system may be written in various versions of various languages. In some embodiments, a localized computer program or cloud-based software system comprises one sequence of instructions. In some embodiments, a localized computer program or cloud-based software system comprises a plurality of sequences of instructions. In some embodiments, a localized computer program or cloud-based software system is provided from one location. In other embodiments, a localized computer program or cloud-based software system is provided from a plurality of locations. In various embodiments, a localized computer program or cloud-based software system includes one or more software application modules. In various embodiments, a localized computer program or cloud-based software system includes, in part or in whole, one or more web applications, one or more mobile applications, one or more standalone applications, one or more web browser plug-ins, extensions, add-ins, or add-ons, or combinations thereof.

Web Application

In some embodiments, a localized computer program or cloud-based software system includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. A web application for providing a career development network for artists that allows artists to upload information and media files, in some embodiments, includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

Mobile Application

In some embodiments, a localized computer program or cloud-based software system includes a mobile application provided to a mobile digital processing device. In some embodiments, the mobile application is provided to a mobile digital processing device at the time it is manufactured. In other embodiments, the mobile application is provided to a mobile digital processing device via the computer network described herein. It is specifically contemplated that the software application is configured for display on a mobile device and/or a computer screen. In specific instances, the software application is configured to receive inputs via a graphical user interface (GUI). See FIG. 2 for an illustration. In preferred instances, the software application is used to guide a user's choice on sample number to load based on chosen coverage and genome sizes.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

Standalone Software Application

In some embodiments, a localized computer program or cloud-based software system includes a standalone software application or a plurality of software applications, which is a program that is run as an independent computer process, not an add-on to an existing process, e.g., not a plug-in. Those of skill in the art will recognize that standalone software applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a localized computer program or cloud-based software system includes one or more executable compiled software applications.

Software System

The systems, platforms, software, networks, and methods disclosed herein include, in various embodiments, software, server, and database modules. In view of the disclosure provided herein, software system are created by techniques known to those of skill in the art using machines, software, and languages known to the art. The software system disclosed herein are implemented in a multitude of ways. In various embodiments, a software system comprises a file, a section of code, a programming object, a programming structure, or combinations thereof. In further various embodiments, a software system comprises a plurality of files, a plurality of sections of code, a plurality of programming objects, a plurality of programming structures, a plurality of software applications, or combinations thereof. In various embodiments, the one or more software system(s) comprise, by way of non-limiting examples, a web application, a mobile application, and a standalone software application or in combination of a plurality of any of the abovementioned. In some embodiments, software system is in one computer program or application. In other embodiments, software system is in more than one computer programs or applications. In some embodiments, software system is hosted on one machine. In other embodiments, software system is hosted on more than one machines. In further embodiments, software system is hosted on cloud computing platforms. In some embodiments, software system is hosted on one or more machines in one location. In other embodiments, software system is hosted on one or more machines in more than one locations.

Targeted Detection by a User

The present disclosure contemplates integration of an automation workflow with a software system. See, e.g., FIG. 1. In many aspects, a user accesses an interface to make a selection of at least one target for analysis. The target can be one or more nucleic acid sequences derived from a reference genome from a biological creature (e.g., a virus, a bacterium, a yeast, a fungi, or another multi-cellular organism) comprising a nucleic acid sequence.

In many aspects, the disclosure contemplates software applications where a user can choose a coverage depth needed for a genome. The term breadth of coverage as used herein, describes a relation between sequence reads and a reference (e.g. a whole genome or a locus), unlike sequencing depth which describes a total read number. The average depth of sequencing coverage can be defined theoretically as LN/G, where L is the read length, N is the number of reads and G is the haploid genome length. The breadth of coverage is the percentage of target bases that have been sequenced for a given number of times.

In some instances, a user can select a coverage depth for analyses from one or more of a variety of different biological creatures. FIG. 2, for example, illustrates an exemplary user interface. As shown on FIG. 2, a user (e.g., a customer) can choose a genus and a desired coverage depth range. A software application of the disclosure then proceeds to calculate the pooling volume for each sample to achieve the chosen coverage range, based on sequences obtained for each species from a reference genome. The software then proceeds to pass this information to the robotics apparatus for sample library pooling control. For each selected target, the software application auto-fills the genome size based on a reference genus size. This prevents overloading the flow cell with too many samples by blocking addition of any new samples once at capacity. As illustrated on FIG. 2, this structure also allows for the detection of multiple target sequences organisms within the same flow cell.

In some instances, a user can choose a genus and a coverage depth for detecting at least one nucleic acid derived from a microorganism of the Escherichia genus, the Listeria genus, the Salmonella genus, or the Campylobacter genus. See, e.g., FIG. 2. In other cases, at least one target nucleic acid is derived from a plurality of nucleic acids selected from, but not limited to, the microorganism of the Bordetella genus, Chlamydophila genus, Mycoplasma genus, Klebsiella genus, Pseudomonas genus, Enterobacter genus, Citrobacter genus, Yersinia genus, Neisseria genus, Bacillus genus, Streptococcus genus, Staphylococcus genus, Enterococcus genus, Clostridioides genus, Proteus genus, Acinetobacter genus and Mycobacterium genus.

In yet other cases, a user can select a target nucleic acid region for analyses from one or more of the viruses consisting of SARS-CoV-2, influenza A, influenza B, Human Respiratory Syncytial Virus (RSV). In other cases, at least one target nucleic acid is derived from a plurality of nucleic acids selected from the viruses consisting of, but not limited to, adenovirus, coronavirus 229E, coronavirus HKU1, coronavirus NL63, human metapneumovirus, human rhinovirus/enterovirus, parainfluenza virus 1, parainfluenza virus 2, parainfluenza virus 3, parainfluenza virus 4, Filovirus, Paramyxovirus, Retrovirus, Flavivirus, Bunyavirus, Poxvirus and Lyssavirus.

Non-limiting examples of specimens from the Coronavirus genus that can be distinguished with the methods of the disclosure include both viruses with low case fatality rate (CFR), namely HCoV-NL63, HCoV-229E, HCoV-OC43, and HCoV-HKU1, and those with high CFR, namely, MERS-CoV, SARS-CoV, and SARS-CoV-2. Non-limiting examples of microorganism of the Salmonella genus include Salmonella Enteritidis, Salmonella Typhimurium, Salmonella Newport, Salmonella Javiana, Salmonella Infantis, Salmonella Montevideo, Salmonella Heidelberg, Salmonella Muenchen, Salmonella Saintpaul, Salmonella Oranienburg, Salmonella Braenderup, Salmonella Paratyphi B var. L(+) Tartrate+, Salmonella Agona, Salmonella Thompson, and Salmonella Kentucky. Non-limiting examples of microorganism of the Escherichia genus include E. coli 0103, E. coli 0111, E. coli 0121, E. coli 0145, E. coli 026, E. coli 045, and E. coli 0157. Non-limiting examples of Listeria genus include L. monocytogenes, L. grayii, L. weishimeri, L. marthii, L. innocua, L. ivanovii, L. seeligeri. Non-limiting examples of microorganisms of the Campylobacter genus include C. jejuni, C. lari, or C. coli.

In yet other embodiments, the disclosure contemplates applying the systems disclosed herein to the detection of sexually transmitted disorders (STDs). More than 30 different bacteria, viruses, and parasites can be transmitted through sexual activity. Bacterial STIs include Chlamydia, gonorrhea, and syphilis. Viral STIs include genital herpes, HIV/AIDS, and genital warts. Parasitic STIs include trichomoniasis. In some instances, the disclosure contemplates detection of a specimen that is associated with a sexually transmitted disease. In some instances, the specimen is selected from the group consisting of bacterial vaginosis, Chlamydia, gonorrhea, genital herpes, hepatitis, HIV/AIDS, human papillomavirus (HPV), pelvic inflammatory disease (PID), syphilis, trichomoniasis, and other STDs. Non-limiting examples of sexually transmitted infections where the processes of the disclosure can be applied include Chlamydia, gonorrhea, Hepatitis B virus (HBV), Herpes simplex virus type 2 (HSV-2), Human immunodeficiency virus (HIV), Human papillomavirus (HPV), Syphilis, Trichomoniasis, Mycoplasma genitalium, Urinary Tract infections and wound infections. Non-limiting examples of pathogens causing those infections include Chlamydia trachomatis, Neisseria gonorrhoeae, Hepatitis B virus (HBV), Herpes simplex virus type 2 (HSV-2), Human immunodeficiency virus (HIV), Human papillomavirus (HPV), Treponema pallidum, Trichomonas vaginalis, Mycoplasma genitalium, Providencia stuartii, saprophyticus agalactiae, Aspergillus flavus, dubliniensis, Candida parasilosis, Trichosporon asahii, Trichosporon beigelii or Staphylococcus aureus.

Other Definitions

Unless defined otherwise, all technical and scientific terms used herein have the same meaning as commonly understood by one of ordinary skill in the art to which this invention belongs. For purposes of interpreting this specification, the following definitions will apply and whenever appropriate, terms used in the singular will also include the plural and vice versa.

Note that as used herein and in the appended claims, the singular forms “a,” “an,” and “the” include plural referents unless the context clearly dictates otherwise.

As used herein, “bioinformatics pipelines” are used for biologic data analysis.

As used herein, “software system” is implemented in robotic control and automation of mechanical parts, as well as communication pathways between a plurality of software applications networked via a plurality of respective APIs within the software system. Execution of software applications can be achieved via code-based or GUI input.

The term “coverage” or “sequencing coverage” as used herein, describes a relation between sequence reads and a reference (e.g. a whole genome or a locus), unlike sequencing depth which describes a total read number. The average depth of sequencing coverage can be defined theoretically as LN/G, where L is the read length, N is the number of reads and G is the haploid genome length.

The term “breadth of coverage” or “breath of sequencing coverage” as used herein is the percentage of target bases that have been sequenced for a given number of times. Depth of coverage can be affected by the accuracy of genome alignment algorithms and by the uniqueness or the ‘mappability’ of sequencing reads within a target genome.

Where a range of values is provided, e.g., range of short nucleic acid sequencing reads (e.g., 1×75 bps, 2×150, and 2×300 bps) and long nucleic acid sequencing reads (of the order of kb), it is understood that each intervening value, between the upper and lower limit of that range and any other stated or intervening value in that stated range is encompassed within the invention. The upper and lower limits of these smaller ranges may independently be included in smaller ranges, and are also encompassed within the invention, subject to any specifically excluded limit in the stated range. Where the stated range includes one or both of the limits, ranges excluding either or both of those included limits are also included in the invention.

### Examples

The following examples are put forth so as to provide those of ordinary skill in the art with a complete disclosure and description of how to make and use the present invention and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example 1: Integration of an Automation Workflow with a Software System-Bioinformatics Pipelines

Software Application: Wizard

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein.

(Wizard) Software Application for Sample to Plate Mapping and Final Pooling Volumes

A software system was created to actuate the automation (physical) workflow of nucleic acid sequencing preparation. A software application was also created to deploy the workflow that informs the library pooling volumes, data analysis, and data report. The software application deployed by the software system is illustrated in FIG. 2. In this instance, a sample was received in Applicants Clinical Laboratory Improvement Amendments (CLIA) laboratory.

The software application provided a user with a list of pre-defined bacteria (at the genus level), for which there is a pre-determined “representative” genome size in bp. The user chose from 1 of 3 bins of coverage to apply to that given sample. The bins ranged from, but were not limited to, 10-20×, 20-30×, and 30-40×. Based on these choices, the number of required base-reads per sample was calculated by the software application. The software application assigned each sample to a plate well position as they are added. The user continued to add samples with coverages to the run configuration while the sum of all required base-reads was equal to or lower than the average sequencing capacity of the flow cell (say 1.1 Gb, for example), after which point the flow cell is considered to be at maximum capacity.

Because more molecules loaded will “grab” more of the total reads, and since some samples with large genomes at high coverage will require more reads than small genomes at low coverage, the library pooling volumes per sample may be variable. The software application calculated the pool volume for each library to ensure each sample receives the correct percentage of the total base-reads to achieve the targeted coverage. Specifically, the average sequencing output capacity of the sequencing flow cell was divided amongst all sample libraries in the sequencing pool. The number of base-pairs (gathered as 2×150 bp short reads, for example) assigned to a given sample was determined by the fraction of molecules in the pooled library. Coverage refers to the number of base-calls at each base-position along an entire genome: 30× coverage means that, on average, each base-position of DNA has 30 base-calls assigned to that position from the collection of short reads that come from the sequencer. Therefore, for a given genome of size N (in base pairs), to obtain X coverage, (N*X) bases were assigned from the entire flow cell output to this given sample to reach the target coverage of X. Fewer base-reads than this number resulted in less than the target coverage.

In sum, the software application calculates the pooling volume for each sample to achieve the chosen coverage range. See, e.g., pooling volumes based on software application (Wizard) calculations to meet the coverage chosen per sample 103. The software application actuated the Automated Liquid Handling System for moving the calculated library pooling volumes into the sequencer.

Integration of an Automation Workflow with a Software System

Off-Deck Work (Off Workstation)

Enrichment

A sample was received in Applicants Clinical Laboratory Improvement Amendments (CLIA) laboratory. The sample was added to a growth media to enrich and/or isolate for one or more biological organism(s). In this example, the media was selected to enrich for Salmonella, Campylobacter, Listeria, and/or E. coli. This biological culture was subsequently subsampled, transferred into a resuspension buffer, and aliquoted onto a sample plate suitable for processing by the automated liquid handling workstation. A user accessed a GUI for initiating the on-deck automated processing. The user placed a volume of this suspension into a well of the plate onto the deck of a system of the disclosure.

Preparing the Reagent Cartridge for the Sequencer

Sequencing cartridge was thawed at 2-8° C. or room temperature as per manufacturer's recommendation. Once thawed, the reagent cartridge was inverted 10 times to mix, and then visually inspected to verify that all positions are completely thawed and free of precipitates. The sequencing flow cell was placed into the thawed cartridge and then the thawed reagent cartridge was placed on the Work Deck at a location at room temperature until the sample was ready to be loaded.

On-Deck Work (Workstation)

An automated multiple-assay sample-preparation and extraction platform for regulated bioanalysis was developed by integrating a software system, comprising a plurality of software applications networked together via a plurality of APIs, (see FIGS. 1 and 2) with a Hamilton STAR robotic liquid handler. The software system was programmed and combined with all associated study and extraction specific variables and information (including a LIMS worklist) into a database file that was imported into the liquid-handling workstation's method. The Hamilton Microlab STAR's (Hamilton Robotics, Reno, NV, USA) liquid-handling method was programmed using a specific software application and executed all sample-preparation commands for each extraction module. Various protocols were programmed within the software application and successfully executed. The exemplary protocol tested on this example was written to execute the flowing steps:

Interface

Upon entry of the desired target nucleic acids to be analyzed, in this case, Salmonella, Campylobacter, Listeria, and E. coli., the software application calculated (based on genome size and coverage) a capacity bar for each sample. The software application was pre-programed with select genome sizes and the genome size was pre-programmed to autofill based on the genus. This system prevented the overloading of the flow cell, and provided an easy visual of the flow cell capacity. See FIG. 2. See Example 1.

Lysis on Deck (Module A)

The resuspended isolate solution contained in a 96 well processing plate was placed onto the liquid handling workstation for processing. The liquid-handling workstation transferred about 40 μL of supernatant to a 96-well processing plate for lysis. Liquid handling manipulations using the liquid-handling workstation employed the use of 50-μL tips found in roll 303, depending on the run. Lysis solution was then added to each sample in sample plate 310. Liquid handling manipulations using the liquid-handling workstation employed the use of 300-μL tips found in row 304, depending on the run. The liquid handling system pipetted the mixtures up and down. The cell solution mixed in lysis buffer was incubated for 55° C. for a first period-of-time. Subsequently, the mixture was incubated at 75° C. for a second period-of-time.

SPRI beads were added to samples in the ratio of 0.65:1 was added to sample well on processing plate 314. The liquid handling system pipetted the mixture up and down and incubated the sample/SPRI mixture for another period-of-time. Subsequently, the liquid-handling workstation automatically transferred the entirety of the supernatant to a 96-well processing in the SPE plate 329 for a period-of-time. Liquid handling manipulations using the liquid-handling component within the workstation were employed to aspirate the supernatant.

Liquid handling manipulations using the liquid-handling workstation employed the use of 300 μL tips and 50 μL tips for nucleic acid extraction and isolation. To this cleaned up sample, SPRI beads were automatically added for size selection (0.65:1) via the liquid handling system. The liquid handling system automatically pipetted this mix up and down, thus generating a homogenized SPRI-sample mix. The SPRI sample mix was incubated at room temperature for another period-of-time. Subsequently, to this washed SPRI-sample mix was added to a magnet for another period-of-time.

While on the magnet, the liquid handling system automatically aspirated and discarded the supernatant. The samples attached to the beads were washed with two sequential 80% EtOH washes, in this case an EtOH wash solution that the liquid handling system was programmed to retrieve from 305, 306, or 307 depending on the configuration of the apparatus. The liquid handling system retrieved an elution buffer, which could have been placed on either reagent cartridge 302, 305, or 306 depending on the configuration, and eluted the samples from the beads.

Tagmentation on Deck and PCR (Module B)

The sample, eluted from beads, was transferred via the liquid handling system into a new 96-well plate within a thermal cycler 313 for tagmentation. The liquid handling system transferred a mix of tagmentation enzyme and buffer into the 96-well plate placed on the thermal cycler. The lid of the thermal cycler was automatically closed upon loading and ran at 55° C. for a second period-of-time with a programmed hold of 25° C. after the 55° C. period-of-time ended.

Tagmentation Stop and Wash

At the end of the tagmentation period, the liquid handling system pipetted a reaction quenching solution to end the tagmentation to each sample and then placed the SPE plate back into the thermal cycler to further deactivate the tagmentation enzyme. Immediately following the termination of the tagmentation reaction, the samples are placed onto the magnet and undergo 3 rounds of washing all while being functionally connected to the magnet. The sample plate was transferred away from the magnet and the samples were then resuspended directly in PCR reaction solution, combined with a unique sequencing adapter pair and placed into the thermal cycler for amplification of Salmonella, Campylobacter, Listeria, and E. coli genomes.

Pooling and Library Nucleic Acid Size Selection and Dilution

A set volume of each of the samples was pooled and added into a 0.5 tube on rack. The software application was used to calculate the pooling volume based on the coverage depth selected by a user for each genome. After pooling individual samples based on user defined parameters, the sample pool was transferred into a new sample plate. 70 μL of the samples were with 42 μL of SPRI beads (ratio of 0.65×), mixed and incubated for a period of time prior to being transferred robotically to a magnet. On the magnet the samples were incubated for another period of time, then while being functionally connected to the magnet, the supernatant was discarded and the sample was washed with 80% EtOH twice. The sample plate was removed from the magnet and the sample was eluted in Tris buffer. 80 μL of the sample was transferred into a new sample well position on the plate.

The automated liquid handling platform performed another SPRI size selection to remove all large fragments from the sample by adding 36 μL of SPRI to the 80 μL of sample (0.45 ratio). The combined SPRI/Sample mixture plate was transferred robotically to the magnet, incubated for a period of time and 100 μL of supernatant was transferred into a new sample well and then the plate was transferred off the magnet position to a suitable carrier. 50 μL of SPRI was robotically added to the sample to capture all remaining nucleic acids in solution. The sample was mixed, incubated and returned to the magnet position. While being functionally connected the instrument removed and discarded the supernatant, and washed the sample with 80% EtOH twice. The sample was air-dried on the magnet position. Then was eluted with 80 μL of TRIS buffer and 70 μL was transferred to a new well position.

A final SPRI library clean up step was performed by the automated platform by adding and mixing 45 μL of SPRI to the 70 μL of sample previously transferred into a new well on the plate. The solution was incubated for a period of time, then the plate is transferred to the magnet position. After another period of time, the liquid handling system removed and discarded the supernatant and then washed the sample twice with 80% EtOH while the plate was functionally connected to the magnet. The sample was allowed to dry for a period-of-time, then eluted with 50 μL of TRis Buffer.

The liquid handling system diluted the final library in Tris to the desired and combined it with sequencing control at a range optimal for sequencing on the chosen sequencing platform. The experimental steps described above were performed on a work-deck illustrated in FIGS. 3A-3F and a sequencing cartridge was loaded with nucleic acids prepared for sequencing-by-synthesis as shown in FIGS. 4A-4F.

Loading the Sequencing Module

Using a clean 300 μL pipette tip, the liquid handler pierced the foil cover over the loading port labeled “Load Sample” on a sequencing-by-synthesis cartridge. 20 μL of the pooled DNA library was loaded into the “Load Sample” reservoir via the loading port into the sequencing cartridge, subsequently onto the sequencing flow cell, on the stage. Subsequently a robotic arm grabbed the sequencing cartridge and loaded it into the sequencing instrument 315 as shown in FIG. 4G-4J. All robotic movements were actuated by the software application. To the best of our knowledge, this work represents the first complete execution of a sample preparation to sequencing, including loading of nucleic acid into a flow cell following by subsequent loading of the sequencing cartridge into a sequencing-by-synthesis sequencer.

### Example 2: Integration of an Automation Workflow with a Software System-Bioinformatics Pipelines in the Detection of Salmonella enterica, Listeria monocytogenes, and Escherichia coli

Software Application: Wizard Software

Various clinical samples, as well as surface swaps of surfaces from the clinical environment was received in Applicants Clinical Laboratory Improvement Amendments (CLIA) laboratory for analysis. The clinical laboratory, a customer, requested the analysis of the samples for Salmonella enterica, Listeria monocytogenes, and Escherichia coli.

In this case, the clinical samples were isolated for the organisms to be analyzed by adding the samples to culture media designed to support the growth of for Salmonella enterica, Listeria monocytogenes, and Escherichia coli. The samples were cultured in solid medium overnight. The next day, this biological isolate was subsequently picked, resuspended in tris buffer. A user accessed a graphical use interface (GUI) for initiating the on work-deck automated processing. A user initiated the fully automated program by turning on the system (including software system) and loading the samples into a chamber of the system. The user placed a volume of this resuspended cellular isolate solution into wells of a plate onto the deck of a system of the disclosure.

The user selected Salmonella enterica, Listeria monocytogenes, and Escherichia coli as the organism to be analyzed and the software application populated the genome size for each organism. The user then selected 30-40× as coverage for S. enterica, 10-20× as coverage for L. monocytogenes, and 30-40× for E. coli as coverage for E. coli. Based on these choices, the number of required base-reads per sample was custom calculated by the software application. The software application assigned each sample to a plate well position as they are added. The user continued to add samples with coverages to the run configuration while the sum of all required base-reads was equal or lower to the predetermined sequencing capacity of the flow cell (say 1.1 Gb, for example), after which point the flow cell is at maximum capacity. The software application calculated the pool volume for each library to ensure each sample receives the correct percentage of the total base-reads to achieve target coverage. Specifically, the average sequencing output capacity of the sequencing flow cell was divided amongst all sample libraries in the sequencing pool. The number of base-pairs (gathered as 2×150 bp short reads) assigned to a given sample was determined by the fraction of molecules in the pooled that comes from a library, where more molecules took up more of the total base-reads available. Coverage referred to the number of base-calls at each base-position along an entire genome: 30× coverage means that, on average, each base-position of DNA has 30 base-calls assigned to that position from the collection of short reads that come from the sequencer. Therefore, for a given genome of size N (in base pairs), to obtain X coverage, (N*X) bases were assigned from the entire flow cell output to this given sample to reach the target coverage of X. Fewer base-reads than this number resulted in less than the target coverage.

In sum, the software application calculated the pooling volume for each sample to achieve the chosen coverage range. See, e.g., pooling volumes based on software application wizard calculations to meet the coverage chosen per sample 103. The integration of the software system allowed the system to actuate the Automated Liquid Handling System for moving the calculated library pooling volumes into the sequencer.

The remaining protocol, including the preparation of the reagent cartridge for the sequencer, the on work-deck (workstation) workflow, the steps performed in Module A, Module B, and Module C, and the loading of the samples into the sequencing cartridge, and subsequently onto the sequencing flow cell, was prepared substantially in the same manner as described in Experiment 1. In parallel, a manual experiment was conducted to compare and contrast the performance of the fully automated system to a manual performance, the results of which are outlined in TABLE 1.

In both instances (manual and automated), the identification of the microorganism was confirmed with standard serotyping experiments and the average % of reads aligned to expected microorganism was correlated. Early results suggest that the average sequencing coverage could be higher for some organisms, although more experiments need to be repeated to improve the calculation of standard deviations. A summary of the results is outlined below in Table 1

TABLE 1

| | | | | | % of | Average | | |
|---|---|---|---|---|---|---|---|---|
| | | | | | samples | % of | | Average |
| | | | | Average | with | Reads | Average | Median |
| | | Sample | | Raw | correct | correctly | Seq. | Insert |
| Organism | Type | Count | Method | Qscore | serotype | aligned | Coverage | Size |
| Salmonella | Gram− | 6 | Manual | 35.0 ± 0 | 100% | 99.99% ± 0 | 43.3 ± 5.83 | 370.33 ± 2.06 |
| enterica | | | Automated | 35.5 ± 0.55 | 100% | 99.99% ± 0 | 46.7 ± 12.01 | 332 ± 1.21 |
| Listeria | Gram+ | 8 | Manual | 35.0 ± 0 | 100% | 99.99% ± 0 | 42.7 ± 8.09 | 380.66 ± 4.89 |
| monocytogenes | | | Automated | 35.0 ± 0 | 100% | 99.99% ± 0 | 44.13 ± 17.64 | 402.63 ± 2.13 |
| Escherichia | Gram− | 3 | Manual | 36.0 ± 0 | 100% | 99.99% ± 0 | 59.0 ± 8.29 | 321.0 ± 11.01 |
| coli | | | Automated | 35.0 ± 0 | 100% | 99.99% ± 0 | 70.6 ± 20.22 | 369.05 ± 2.64 |

### Example 3: Integration of an Automation Workflow with a Software System-Bioinformatics Pipelines in the Detection of a Variety of Viral Species, Including the Delta Variant of SARS-CoV-2 and the Omicron Variant of SARS-CoV-2

Various clinical samples (respiratory specimens) were received in Applicants Clinical Laboratory Improvement Amendments (CLIA) laboratory for analysis. A customer, requested the analysis of samples of Delta and the Omicron variants of SARS-CoV-2 virus.

RNA Extraction: The analysis starts with RNA (in elution buffer), extracted from respiratory specimens, either using MagMAX Viral RNA Isolation Kit (catalog #AM1939) through the manual workflow or using MagMAX Viral/Pathogen Nucleic Acid Isolation Kit (Catalog #A42352 from Thermo Fisher Scientific) through the automated workflow on KingFisher Flex Purification system with 96-Deep well head (Catalog #5400630 from Thermo Fisher Scientific), as recommended by the manufacturer. No enrichment step was performed for the virus analyses.

A user accessed an interface (GUI) for initiating the on work-deck automated processing. A user initiated the fully automated program by turning on the system (including software system) and providing the samples into a chamber of the system. The user placed a volume of this suspension into a plate onto the deck of a system of the disclosure. The automated analyses started with synthesizing cDNA from the extracted RNA for each of the samples loaded into the wells of the plate in independent reverse transcription reactions on the work-station. Then the viral target amplicons were captured from the synthesized cDNA through a multiplex PCR process using a panel of barcoded target capture primers. After this “Target-capture” PCR step, there was a Solid Phase Reversible Immobilization (SPRI) bead-based cleanup during which all the excess primers and any short amplification products were removed. All of these steps were automatically performed on the work-deck and control by the software program of the disclosure. Following this, the amplicons were subjected to another round of PCR, termed as “Barcoding PCR”, where the second set of barcodes are added to the amplicons using the rapid library primers from ONT. Following this step, the dual barcoded amplicons from all the samples were pooled together and cleaned up through another SPRI bead process. After this step, the ONT sequencing adapters were ligated to all the barcoded amplicons. The samples were automatically loaded into a pore sequencer as described in for example, U.S. Pat. No. 10,597,714B2, U.S. Pat. No. 11,282,587 B2 (incorporated here by reference in their entireties), and then sequenced on the MinION sequencer.

The bioinformatics pipeline, performed a series of steps including demultiplexing, error correction and alignments on the sequencing reads of the amplicons to make the detection calls. The bioinformatics detection algorithm took the relative ratios of the sequencing signal for SARS-CoV-2 primers, the internal PCR control, and the human housekeeping gene into account to make an invalid/positive/negative call. Samples with insufficient total read coverage were classified as invalid. The remaining samples had their SARS-CoV-2 signal compared to empirically derived thresholds. These thresholds distinguished true SARS-CoV-2 signal from noise. Each primer had its own threshold and the pipeline leverages the redundancy of SARS-CoV-2 specific primers to make a call.

The results are outlined below on TABLE 2.

TABLE 2

| | | | | | % of | | % of | |
|---|---|---|---|---|---|---|---|---|
| | | | | | samples | | Samples | |
| | | | | | with | | with | |
| | | | | Average | correct | Average | 0 | Average |
| | | Sample | | Raw | variant | Assembly | VADR | Read |
| Organism | Variant | Count | Method | Qscore | identification | Coverage | Alerts | Length |
| SARS- | Delta | 4 | Manual | 14.1 ± 3.0 | 100% | 99.35% ± 0.02 | 100% | 635.95 ± 286.96 |
| CoV-2 | | | Automated | 13.3 ± 3.4 | 100% | 99.11% ± 0.19 | 100% | 588.76.95 ± 271.23 |
| SARS- | Omicon | 54 | Manual | 14.2 ± 3.1 | 100% | 97.78% ± 3.94 | 100% | 633.95 ± 274.11 |
| CoV-2 | | | Automated | 13.5 ± 3.0 | 100% | 97.78% ± 2.58 | 100% | 591.71 ± 264.19 |

### Example 4: Integration of an Automation Workflow with a Software System-Bioinformatics Pipelines in the Detection of a Variety of Sexually Transmitted Diseases (STDs)

Sexually transmitted infections (STIs) continue to cause a significant burden of disease globally. Besides the high incidence of STIs, several causative bacteria are becoming resistant to or have low susceptibility to antibiotics, including multidrug resistant (MDR) Neisseria gonorrhoeae and Mycoplasma genitalium. As recently as 2023, it has been reported that two N. gonorrhoeae strains have broken through all current commercially available antibiotic options, posing an urgent threat. Many individuals with STI carry multiple pathogens, who are either asymptomatic and/or latent carriers and are not tested for all pathogens. The present disclosure contemplates that various clinical samples can be analyzed for STD's by a fully automated system of the disclosure as described in Examples 1-3.

Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

We claim:

1. A system for automating a nucleic acid sequencing process, the system comprising:
a robotic arm having a left effector and a right effector, said robotic arm connected to a rotary axis, said rotary axis connected to an apparatus for actuating one or more programmed movements of the robotic arm, wherein the apparatus is configured for receiving an input from a fully automated software system for:
moving said robotic arm towards a sequencing module, comprising a sequencing cartridge and sequencing flow cell, on a location;
extending the left effector and the right effector of the robotic arm around the sequencing module on the location and subsequently contracting said effectors around the sequencing module, thereby holding the sequencing module with the robotic arm;
moving the sequencing module that is held by the robotic arm into a sequencer thereby loading the sequencing module; and
a sequencer for sequencing a sample or a plurality of samples in the sequencing flow cell of the sequencing module;
whereby the system is controlled by the fully automated software system.

2. The system of claim 1, wherein the rotary axis is configured for rotating 360°.

3. The system of claim 1, wherein the rotary axis rotates the robotic arm above the location housing the sequencing module, comprising a sequencing cartridge and sequencing flow cell.

4. The system of claim 1, wherein the left effector and the right effector are symmetrical.

5. The system of claim 1, wherein the left effector and the right effector are configured for lifting at least 1 pound, at least 2 pounds, at least 5 pounds, or at least 10 pounds.

6. The system of claim 1, wherein the sequencer is configured for performing sequencing-by-synthesis.

7. The system of claim 1, wherein the sequencer is configured for receiving one or more inputs directly from the system.

8. The system of claim 1, wherein the sequencer automatically starts a sequencing run upon loading of the sequencing module, comprising a sequencing cartridge and sequencing flow cell.

9. The system of claim 1, where the system further comprises a liquid handling system functionally connected to a multi-module workstation.

10. The system of claim 9, where the liquid handling system is configured for piercing a cover of the loading port of a sequencing cartridge.

11. The system of claim 10, where the liquid handling system is configured for loading the pierced sequencing module, comprising a sequencing cartridge and sequencing flow cell, with a sample or a plurality of sample prepared for sequencing.

12. The system of claim 11, where the sample or a plurality of sample was prepared for sequencing in the multi-module workstation.

13. The system of claim 12, where the sample or a plurality of sample was robotically prepared for sequencing in the multi-module workstation without human intervention.

14. The system of claim 9, where the workstation comprises a plurality of multi-well plates for receiving samples.

15. The system of claim 9, where the workstation comprises a plurality of racks.

16. The system of claim 15, where the plurality of racks include racks for holding reagents for extracting a nucleic acids from a cell.

17. The system of claim 16, where the reagents for extracting the nucleic acids from the cell include reagents for cell lysis.

18. The system of claim 15, where the plurality of racks include chambers for holding reagents for fragmentation of a nucleic acids.

19. The system of claim 15, where the plurality of racks include chambers for holding reagents for end repair of a nucleic acids.

20. The system of claim 15, where the plurality of racks include chambers for holding reagents for reverse transcription of a nucleic acids.

21. The system of claim 15, where the plurality of racks include chambers for holding reagents for amplification of one or more nucleic acids.

22. The system of claim 21, where the plurality of racks include chambers for holding reagents for indexing of one or more nucleic acids.

23. The system of claim 15, where the plurality of racks include chambers for holding reagents for adding a sequencing adaptor or a plurality of sequencing adaptors to one or more nucleic acids.

24. The system of claim 9, where the workstation comprises a magnet plate.

25. The system of claim 9, where the workstation comprises a vacuum manifold.

26. The system of claim 9, where the workstation comprises a thermal cycler.

27. The system of claim 9, where the workstation is an open workstation.

28. The system of claim 1, wherein the fully automated software system comprises a plurality of software subsystems, software applications, software components, and software modules networked together.

29. The system of claim 28, where in the fully automated software system comprises a plurality of External APIs.

30. The system of claim 28, where in the fully automated software system comprises a plurality of internal APIs.

31. The system of claim 28, where in the fully automated software system comprises a plurality of AI software applications and systems.

32. The system of claim 28, where in the fully automated software system comprises a plurality of bioinformatics pipelines.

33. The system of claim 28, where in the fully automated software system comprises a plurality of analytical and reporting softwares or modules.

34. The system of claim 1, wherein the fully automated software system comprises a plurality of software applications networked together.

35. The system of claim 9, wherein the fully automated software application network is integrated with a bioinformatics pipeline and a software application provides input to the liquid handling system, based on parameters determined by software application to meet desired output parameters from the bioinformatics pipeline functionally connected to the multi-module workstation.

36. The system of claim 35, wherein the input is a sample pooling volume for a sequencing library.

37. A software application comprising instructions which, when the program is executed by a computer, cause the computer to instruct the system of claim 1 to robotically insert a sequencing module, comprising a sequencing cartridge and sequencing flow cell, into a sequencer.

38. A process for robotically inserting a cartridge into a sequencer comprising using a robotic system of claim 1.
